(19) United States
(12) Patent Application Publication
(10) Pub. No.: US 2006/0135574 A1
(43) Pub. Date: Jun. 22, 2006

(54) Novel uses for estrogen beta agonists

(75) Inventors: Mark Day, Newton, PA (US); Heather A. Harris, Phoenixville, PA (US)

Correspondence Address: COZEN O' CONNOR, P. C., 1900 MARKET STREET, PHILADELPHIA, PA, 19103-3508, US

(73) Assignee: Wyeth, Madison, NJ (US)

(21) Appl. No.: 11/304,037

(22) Filed: Dec. 15, 2005

Related U.S. Application Data
(60) Provisional application No. 60/637,144, filed on Dec. 17, 2004.

(51) Int. Cl. A61K 31/428; A61K 31/423; A61K 31/4184

(52) U.S. Cl. 514/367; 514/375; 514/394

(57) ABSTRACT

This invention provides methods for treating cognitive diseases or disorders and symptoms thereof with estrogen beta selective agonists.

### CROSS REFERENCE TO RELATED APPLICATION

The present invention claims benefit of priority from provisional U.S. Patent Application Ser. No. 60/637,144 filed Dec. 17, 2004, which is incorporated herein in its entirety.

### BACKGROUND OF THE INVENTION

This invention relates to the use of estrogen beta agonists (ERβ selective ligands) to treat cognitive diseases or disorders, including those that manifest themselves in other disorders, such as schizophrenia, multiple sclerosis, depression, Parkinson's Disease, stroke, Alzheimer's Disease, and anxiety disorders, and symptoms thereof.

Schizophrenia is a disorder characterized by three distinct symptom clusters. Positive symptoms consist of hallucinations, delusions and paranoia. Negative symptoms include social withdrawal, flat affect, anhedonia and overall decreased motivation. The neurocognitive deficits (i.e., cognitive symptoms) include severe deficits in attention, episodic memory and executive functioning.

Although both males and females are equally prone to develop schizophrenia, key gender differences are observed. Kraeplin (1909-1915) first observed that female schizophrenia patients were significantly older than males at age of first episode and this finding has been reported in excess of 50 studies (Angermeyer M C and Kuhn L 1998 Eur Arch Psychiatry Neurol Sci. 237(6):351-64). Sex differences in terms of age at onset, symptom expression, and course of illness have been consistently demonstrated in patients with schizophrenia (Seeman M V 1982 Can J Psychiatry 27(2):107-12; Goldstein J M 1988 Am J Psychiatry 145(6):684-9; Goldstein J M and Link B G 1988 J Psychiatr Res. 22(2):141-55; Seeman M V and Lang M 1990 Schizophr Bull. 16(2):185-94; Seeman M V 1996 Can J Psychiatry 41(5):263-4). The onset of schizophrenia in females occurs later than it does for males, peaking at menopause when estrogen production ceases. In a clinical study it was observed that a significantly greater proportion of women had late-onset schizophrenia (females=41% vs. males=20%), occurring when estrogen levels were low or are below basal (Hafner H et al., 1988 Schizophr Bull. 24(1):99-113; Angermeyer M C and Kuhn L 1988; Angermeyer M C et al., 1989 Psychol Med. 19(2):365-82; Lindamer L A et al., 1999 J Clin Psychiatry 60(1):61-7; Lindamer L A et al., 2001 Biol Psychiatry 49(1):47-51). Prior to menopause, females respond better to antipsychotic treatment (Jonsson H and Nyman A K 1991 Acta Psychiatr Scand. 83(5):342-6). Female patients have more affective and paranoid symptoms and fewer negative symptoms than male patients. Symptoms in female patients fluctuate with the menstrual cycle with the highest rate of psychosis occurring at pre-menstrual phases when circulating estrogens are low (Angermeyer M C and Kuhn L 1998; Hafer H, Riecher-Rossler A et al., 1993 Psychol Med. 23(4):925-40; Seeman M V 1996). Drug naive females during first onset have low levels of estrogen (Hafner H 2003 Psychoneuroendocrinology 28 Suppl 2:17-54; Riecher-Rossler 2003 Nervenarzt. 74(5)398-405; Angermeyer M C and Kuhn L 1998; Hafner H, Riecher-Rossler A et al., 1993; Seeman M V 1996). Interestingly, estrogen treatment in females, given in conjunction with antipsychotic medication, improves positive and negative symptoms and reduces extrapyramidial side effects (EPS) liability compared with antipsychotic treatment alone (Rao M L and Kolsch H 2003 Psychoneuroendocrinology 28 Suppl 2:83-96). For example, women undergoing treatment with antipsychotics who were given exogenous estrogen presented with faster improvement of the positive, negative symptoms (PANSS) and cognitive symptoms (Kulkarni et al., 2004) than those without estrogen (Kulkarni J, Riedel A et al., 2001 Schizophr Res. 48(1):137-44; Kulkarni J, Riedel A et al., 2002 Arch Women Ment Health 5(3):99-104). A recent report demonstrated that 33 young men who were diagnosed with severe schizophrenia had a significant decline in hallucinations and delusions while being administered small doses of estradiol for two weeks. Within five days the patients' scores measuring psychotic symptoms fell from around 60 or 70—classified as severe psychosis—to 20 or 30. (HealthyPlace.com (Feb. 25, 2003) Test Provides Hope for Men with Severe Schizophrenia, at www.healthyplace.com/communities/thought disorders/schizo/news/estrogen.asp, last accessed on Sep. 29, 2004).

Glutamatergic and dopaminergic systems are major transmitter systems thought to be integral to the symptomology observed in schizophrenia. Schizophrenia is proposed to be a disorder of altered synaptic function, and drugs that block the N-methyl-D-asparate (NMDA) sub-type of glutamate receptors in the brain, while inducing psychotic symptoms and behaviors in humans and lab animals, also have a negative effect on synaptic plasticity. Schizophrenic patients have increased subcortical dopamine (DA) activity that is currently treated by D₂ antagonists or partial agonists. Moreover, amphetamine (AMPH) challenge to schizophrenic patients induces an increase in DA as measured with positron emission tomography (PET) at the D₂ receptor and a concomitant transient increase in positive symptoms. Animal studies have demonstrated that estrogen has antidopaminergic properties, reducing the concentrations of dopamine (Dupont A, Di Paolo T et al., 1981 Neurosci Lett. 22(1):69-74) and dopamine D₂ receptor sensitivity in the brain (Hafner H, Behren S et al., 1991 Psychiatry Res. 38(2):125-34). In rodent models of psychosis, AMPH and direct D₂ agonists such as apomorphine are used to induce behaviors associated with the increase in DA, such as climbing. In ovariectomized (OVX) rats, chronic 4-week estrogen treatment attenuates the apomorphine (APO) dopaminergic-associated behaviors. Studies have demonstrated that estradiol benzoate will attenuate apomorphine induced climbing in male mice (Fung Y K et al., 1986 Pharmacol Biochem Behav. 24(1):139-41; Fung Y K et al., 1987 Steroids 49(4-5):287-94). Estrogens have been studied as an adjunctive therapy for schizophrenia or as a standalone treatment. (Seeman M V 1996; Lindamer L A et al., 1997 Psychopharmacol Bull. 33(2):221-8; Hoff, Kremen et al., 2001 Am J Psychiatry 158(7):1134-9; Kulkarni J, Riedel A et al., 2001; Rao M L and Kolsch H 2003).

Research has demonstrated that attentional (Buchanan R W, Strauss M E et al., 1997 Am J Psychiatry 154(3):363-70) and cognitive impairments are associated with the negative and the disorganized symptoms of schizophrenia, thus helping to produce impaired conceptual thought (Menon V, Anagnoson R T et al., 2001 Neuroimage 13(3):433-46; Tek, Gold et al., 2002 Arch Gen Psychiatry 59(2):146-53), culminating in deficits of attention, object naming, working memory and long-term memory storage, and the concomitant slowing of information processing and neural activity. Indeed, at best, treatment with atypical antipsychotic treatment only relieves 20-25% of all the symptoms associated with schizophrenia (Hirsch and Weinberger Eds., Schizophrenia 2003). One third of patients respond minimally to antipsychotic medication, and some fail to respond to any treatment (e.g., negative symptoms, neurocognitive function, depressive features and physical illness) (Liberman R P and Corrigan P W 1992 J Neuropsychiatry Clin Neurosci. 4(2):119-24; Conley R R and Buchanan R W 1997 Schizophr Bull. 23(4):663-74). The situation with the current antipsychotic medication and their lack of efficacy on the associated neurocognitive deficits is unfortunate. Not only are these symptoms present prior to the onset of the illness, improvements are proposed to be associated with the remediation of the negative symptoms and more successful rehabilitation of the patient population. Thus, treatment of the cognitive symptoms of schizophrenia is considered a great unmet medical need.

Further, with regards to cognition, it is well established that mammalian glutamatergic activity plays important roles in several distinct learning and memory processes (Morris R G, Moser, E I et al., 2003 Philos Trans R Soc Lond B Biol Sci. 358(1432):773-86). Hippocampal NMDA receptors are needed for learning, indicating a role for plasticity. They are not required for consolidation or retrieval (Day, M., Langston, R. et al., 2003 Nature 10;424(6945):205-9), while hippocampal alpha-amino-3-hydroxy-5-methylisoxazole-4-propionic acid (AMPA) receptors are required for consolidation and retrieval (Morris et al., 2003). Evidence has shown that estrogen influences hippocampal physiology and morphology (for review, see McEwen B 2002 Recent Prog Horm Res. 57, 357-84).

Estrogens have marked effects on hippocampal synaptic function, increasing hippocampal dendrtic spine density and the number of varicosities that can form multiple synapses with different cells (Segal M, Murphy D 2001 Horm Behav. 40(2), 156-9). With an acute rise in estrogen there is a concomitant increase in NMDA receptors and NMDA receptor-mediated Ca²⁺ signals in the hippocampus (for review see Foy M R 2001 Neurobiology of Learning and Memory (76)239-252; Foy M R, Xu, J et al., 1999 Journal of Neurophysiology (81)925-929; Pozzo-Miller L D, Inoue T et al., 1999 Journal of Neurophysiology (81)1404-1411; Woolley C S 1999 Current Opinion in Neurobiology 9(3) 349-54). In addition, estrogen can influence other synaptic signaling processes, including the balance of protein phosphatase and kinase activity (Sharrow et al., 2002 Neuroscience (113)89-97.). The induction of NMDA-receptor dependent long-term depression (LTD) is impaired at hippocampal CA3-CA1 synapses when estrogen production ceases and chronic estrogen replacement restores this effect (Day and Good, January 2005, Neurobiol Learn Mem., 83(1): 13-21). Zeng et al., reported a forebrain specific calcineurin knockout impaired the induction of LTD and this deficit of hippocampal plasticity was related to impaired acquisition of a spatial working memory task (2001 Cell 107(5) 617-29). Manahan-Vaughan & Braunewell reported that the induction of LTD was facilitated in two strains of rats during exploration of a novel environment (1999 Proc. Nat. Acad. Sci. (USA) 96(15) 8739-44). Xu, Anwyl & Rowan (1998) reported that the exploration of a novel environment induced depotentiation of LTP in the CA1 region (1998 Nature 394(6696) 891-4). Further, McGaughy and Sarter reported that ovariectomized rats showed sustained attentional vigilance relative to control animals in a 5-choice response task (1999 Behavioural Neuroscience (113(6) 1216-32). Furthermore, data has shown that ovariectomized rats were slower to extinguish contextual freezing than estrogen-treated rats (Gupta R R, Sen S et al., 2001 Brain Research (888) 356-365). Thus, in addition to schizophrenia, estrogen beneficially affects cognition. For example, cognitive disorders that manifest themselves in other disorders, such as depression, multiple sclerosis, Parkinson's disease, Alzheimer's disease, stroke, and anxiety are beneficially affected from use of estrogen.

Depression is a mental state of depressed mood characterized by feelings of sadness, despair, and discouragement. Depression includes the normal feelings of “the blues” through dysthymic disorder to major depressive disorder. Dysthymic disorder is a mood disorder characterized by depressed feeling (sad, blue, low), loss of interest or pleasure in usual activities, and at least some of the following: changes in appetite and sleep patterns, lack of energy, low self esteem, poor concentration or decision-making skills, and feelings of hopelessness. In dysthymic disorders, symptoms have persisted for more than two years but are not severe enough to meet the criteria for major depressive disorder. Major depressive disorder is characterized by major depressive episodes, a period of daily depressed mood or loss of interest or pleasure in almost all activities with some combination of the following symptoms: altered appetite, weight, or sleep patterns, psychomotor agitation or retardation, diminished capacity for thinking, concentration, or decisiveness, lack of energy and fatigue, feelings of worthlessness, self-reproach, or guilt, frequent thoughts of death or suicide, plans or attempts to commit the latter (Diagnostic and Statistical Manual of Mental Disorders, 4^{th} ed., American Psychiatric Association, Washington D.C., 1994).

Depressive disorders affect over fifteen percent (15%) of the population. In studies of unipolar and bipolar 11 depression, females were twice as likely as males to exhibit clinical depression. Moreover, sex differences were linked to the type of depression, with unipolar depression more frequent (4:1) in females than males. Women suffering from depression are more likely to be hospitalized and more women suffer from anxiety. Therefore, endocrine factors may not only influence the incidence, but also the expression of depression (Birkhauser M 2002 Maturitas 41 Suppl 1: S3-8). Additional investigation of women over forty (>40) years of age demonstrated that they suffer more from unipolar, rather than bipolar, depression (Kuehner C 2003 Acta Psychiatr Scand 108(3): 163-74). In addition to changes in estrogen during per- and post-menopause, the impact of estrogen modulation and hypogonadism on psychosocial behavior is also observed in perimenstrual dysphoric disorder (PMDD) and post-partum depression. Other studies have shown that estrogen-replacement therapy may have anti-depressant effectiveness for some women. Yet estrogen-replacement therapy, as well as traditional antidepressants such as tricyclic antidepressants, monoamine oxidase and selective serotonin reuptake inhibitors (SSRIs), have a number of unwanted side effects or risks. For estrogen-replacement therapy these risks may include heart disease, stroke, and breast cancer. For traditional antidepressants, undesirable side effects and risks may include drug dependency, insomnia, confusion, tachycardia, hypertension, nausea, diarrhea, anxiety, fatigue, and decreased libido, amongst others.

Multiple sclerosis (MS) is a debilitating neurological disease characterized by a progressive loss of motor and sensory function, which eventually leads to paralysis and death. The primary cause of neurological impairment is demyelination of the central nervous system (CNS) caused by an inflammatory autoimmune response. Thus, in people affected by MS, patches of damage called plaques or lesions appear in seemingly random areas of the CNS “white matter,” which is made up of nerve fibers that are responsible for transmitting communication signals both internally within the CNS and between the CNS and the nerves supplying the rest of the body. At the site of a lesion, the nerve insulating material myelin is lost. Studies have shown that the severity of MS is reduced during pregnancy, suggesting that the increased level of sex hormones reduces the autoimmune response. Liu, H. Y. et al., have shown that estrogen treatment confers protection from experimental autoimmune encephalomyelitis (EAE), which is an animal model for MS (J Neurosci Res 2002 70(2): 238-48).

The neurodegenerative disorder caused by substantia nigra (midbrain) dopamine cell death and which is characterized by symptoms of bradykinesia, rigidity, dyskinesia, and postural instability is known as Parkinson's disease. The most effective symptomatic agent in the treatment of Parkinson's disease is levodopa, which is considered the “gold standard.” However, there are concerns regarding the toxicity and the motor and psychiatric effects of the use of levodopa (Olanow C W et al., 2004 Mov Disord. 19(9): 997; Crosby N et al., 2003 Cochrane Database Sys Rev. (1):CD00368). Amantadine, an antiviral drug, has been used to improve symptoms of Parkinson's disease. Yet a review of six randomized controlled trials of amantadine found insufficient evidence of its efficacy and safety in the treatment of idiopathic Parkinson's disease (Crosby N et al., 2003).

Stroke (also called ischemic stroke, stroke syndrome and cerebrovascular accident) is a condition with sudden onset caused by acute vascular lesions of the brain such as infarction from hemorrhage, embolism, or thrombosis, or a rupturing aneurysm. Typical symptoms reflecting the focus of infarction or hemorrhage include hemiparesis, vertigo, numbness, aphasia and dysarthria. Permanent neurologic damage generally is a result.

Alzheimer's disease is a progressive neurodegenerative disorder of the CNS associated with irreversible cognitive and memory loss characterized by extracellular deposition of the amyloid beta peptide in senile plaques, the appearance of intracellular neurofibrillary tangles, cholinergic deficit, extensive neuronal loss and synaptic changes in the cerebral cortex, hippocampus and other areas of brain essential for cognitive and memory functions. Clinical hallmarks of Alzheimer's disease are progressive impairment in memory, judgment, decision-making, orientation to physical surroundings, and language. It is the most common of all neurodegenerative diseases, accounting for about two-thirds of dementia cases with vascular causes and other neurodegenerative diseases mostly covering the remaining one-third.

There is no cure for Alzheimer disease. Four drugs—Aricept® (donepezil HCl), Exelon® (rivastigmine tartrate), Reminyl® (galantamine HBr) and Cognex® (tacrine)—have been approved by the FDA to treat the symptoms of mild to moderate Alzheimer's. These drugs act by increasing the effects of acetylcholineacetylcholine, a chemical that transmits nerve signals in the brain. The drugs have various side effects for some patients. Yet preclinical data has shown that estrogen is neuroprotective, regenerative, a modulator of Apolipoprotein E (APOE, gene; ApoE, protein; the major genetic susceptibility locus of Alzheimer's disease) and potentially disease modifying.

Estrogen also has been shown to have an anti-anxiety effect (Frye C A and Waff M (2004) Behav Neurosci. 118(2):306-13). Anxiety is a disorder characterized by feelings of apprehension and fear, which are accompanied by physical symptoms that are severe and disabling. Symptoms of anxiety include increased respiration, tachycardia, sweating and tremor. Generally, benzodiazepines are effective in treating anxiety disorders; however, long-term use of these compounds may be limited because of associated risks for dependency. See, e.g., R. J. Balderssarini in Goodman & Gilman's The Pharmacological Basis of Therapeautics, 10^{th} ed., 19 (J. C. Hardman & L. E. Limbird eds., McGraw-Hill, 2001).

Two forms of the estrogen receptor have been identified, ERα and ERβ. ERβ is expressed in both male and female rat brain regions (Zhang J Q, Cai, W Q et aL., 2002 Brain Res 935(1-2): 73-80). Distribution of ERβ mRNA and receptors in rodents matches that seen in humans and non-human primates. Also, the consequences and subtleties of the different conformations the receptors adopt when binding ligands have been recently revealed. See U.S. Pat. No. 6,794,403 and EP-A-1451165, which are herein incorporated by reference in their entireties.

A large number of compounds have been described that either mimic or block the activity of 17β-estradiol. Compounds having roughly the same biological effects as 17β-estradiol, the most potent endogenous estrogen, are referred to as “estrogen receptor agonists”. Because the ERβ receptor is located on the oligodendrocytes and on the inner and outer layer of the myelin sheath of the CNS, ERβ agonists may be effective in the treatment of MS. It now has been found that ERβ selective agonists can effect beneficially diseases or disorders with cognitive deficits, such as MS, and alleviate the undesirable symptoms and side effects thereof as described above. This invention is directed to these, and other, important ends.

### SUMMARY OF THE INVENTION

The present invention provides methods for treating Parkinson's disease or symptoms thereof that include the administration of an ERβ selective agonist. The present invention further provides methods for ameliorating symptoms of cognitive diseases or disorders, such as schizophrenia, multiple sclerosis, depression, stroke, Alzheimer's disease and anxiety, which include the administration of an ERβ selective agonist.

In some embodiments of the methods of the invention, the ERβ selective agonist passes the blood-brain barrier or has a longevity in the body that allows for enough accumulation in the brain. In further embodiments, the ERβ selective agonist has one of the Formulas I-XI, infra.

### BRIEF DESCRIPTION OF THE DRAWINGS

FIG. 1: shows three (3) days of estrogen treatment attenuates (top panel) apomorphine induced climbing (AIC) 24 and 48 hours after the last estrogen treatment (middle panel); however, three (3) days of treatment with the ERβ selective ligand, 7-bromo-2-(4-hydroxyphenyl)-1,3-benzoxazol-5-ol, leads to a more profound blockade of AIC (lower panel).

FIG. 2: shows that βERKO mice demonstrate clear hippocampal dependent deficit without any similar amygdale memory deficit.

### DETAILED DESCRIPTION OF THE INVENTION

In one aspect, the present invention provides methods for treating Parkinson's disease comprising the steps of:

a) identifying a patient having Parkinson's disease; and

b) administering to the patient a therapeutically effective amount of an ERβ selective ligand, wherein said ERβ selective ligand is substantially free of ERβ antagonist activity.

In some embodiments, the invention provides methods for ameliorating one or more symptoms or side effects of Parkinson's disease. In further embodiments, the invention provides methods for ameliorating one or more symptoms or side effects of a cognitive disease or disorder such as schizophrenia, multiple sclerosis, depression, stroke, Alzheimer's disease and anxiety.

In some embodiments of the invention, methods are provided for treating Parkinson's disease that comprise identifying a patient having Parkinson's disease and administering to the patient a therapeutically effective amount of an ERβ selective ligand, or a pharmaceutically acceptable salt or prodrug thereof, wherein the ERfl selective ligand has the Formula I: 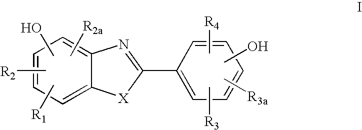
wherein:
R₁ is hydrogen, hydroxyl, halogen, alkyl of 1-6 carbon atoms, trifluoroalkyl of 1-6 carbon atoms, cycloalkyl of 3-8 carbon atoms, alkoxy of 1-6 carbon atoms, trifluoroalkoxy of 1-6 carbon atoms, thioalkyl of 1-6 carbon atoms, sulfoxoalkyl of 1-6 carbon atoms, sulfonoalkyl of 1-6 carbon atoms, aryl of 6-10 carbon atoms, a 5 or 6-membered heterocyclic ring having 1 to 4 heteroatoms selected from O, N or S, —NO₂, —NR₅R₆, —N(R₅)COR₆, —CN, —CHFCN, —CF₂CN, alkynyl of 2-7 carbon atoms, or alkenyl of 2-7 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₂ and R₂ₐ are each, independently, hydrogen, hydroxyl, halogen, alkyl of 1-6 carbon atoms, alkoxy of 1-4 carbon atoms, alkenyl of 2-7 carbon atoms, alkynyl of 2-7 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₃, R₃ₐ, and R₄ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₅ or R₆ are each, independently, hydrogen, alkyl of 1-6 carbon atoms, aryl of 6-10 carbon atoms;
X is O, S, or N R₇;
R₇ is hydrogen, alkyl of 1-6 carbon atoms, aryl of 6-10 carbon atoms, —COR₅, —CO₂R₅ or —SO₂R₅;
or a pharmaceutically acceptable salt or prodrug thereof.

In further embodiments of the invention, the ERβ selective ligand has Formula II: 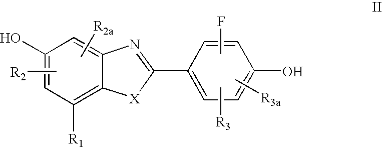
wherein:
R₁ is alkenyl of 2-7 carbon atoms; wherein the alkenyl moiety is optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₂ and R₂ₐ are each, independently, hydrogen, hydroxyl, halogen, alkyl of 1-6 carbon atoms, alkoxy of 1-4 carbon atoms, alkenyl of 2-7 carbon atoms, alkynyl of 2-7 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₃, and R₃ₐ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₅ or R₆ are each, independently, hydrogen, alkyl of 1-6 carbon atoms, aryl of 6-10 carbon atoms;
X is O, S, or N R₇;
R₇ is hydrogen, alkyl of 1-6 carbon atoms, aryl of 6-10 carbon atoms, —COR₅, —CO₂R₅ or —SO₂R₅;
or a pharmaceutically acceptable salt or prodrug thereof.

In some embodiments of the methods of the invention wherein the ERβ selective ligand is of Formula II, or a pharmaceutically acceptable salt or prodrug thereof, X is O. In further such embodiments, X is O and R₁ is alkenyl of 2-3 carbon atoms, which is optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆. In still further such embodiments, the ERβ selective ligand is 2-(3-fluoro-4-hydroxyphenyl)-7-vinyl-1,3-benzoxazol-5-ol or a pharmaceutically acceptable salt or prodrug thereof.

The preparation of ERβ selective ligands having Formulae I and II is described in U.S. Pat. No. 6,794,403 and EP-A-1451165, incorporated herein by reference in their entireties.

In further embodiments of methods of the invention, the ERβ selective ligand is of Formula III: 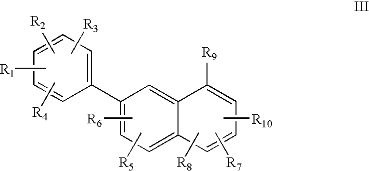
wherein:
R₁, R₂, R₃, and R₄ are each, independently, selected from hydrogen, hydroxyl, alkyl of 1-6 carbon atoms, alkoxy of 1-6 carbon atoms, or halogen;
R₅, R₆, R₇, R₈, R₉, and R₁₀ are each, independently, hydrogen, alkyl of 1-6 carbon atoms, alkenyl of 2-7 carbon atoms, alkynyl of 2-7 carbon atoms, halogen, alkoxy of 1-6 carbon atoms, —CN, —CHO, phenyl, or a 5 or 6-membered heterocyclic ring having 1 to 4 heteroatoms selected from O, N or S; wherein the alkyl or alkenyl moieties of R₅, R₆, R₇, R₈, R₉, or R₁₀ may be optionally substituted with hydroxyl, —CN, halogen, trifluoroalkyl, trifluoroalkoxy, —NO₂, or phenyl; wherein the phenyl moiety of R₅, R₆, R₇, R₈, R₉, or R₁₀ may be optionally mono-, di-, or tri-substituted with alkyl of 1-6 carbon atoms, alkenyl of 2-7 carbon atoms, halogen, hydroxyl, alkoxy of 1-6 carbon atoms, —CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;
   wherein at least one of R₁, R₂, R₃, R₄, R₇, R₈, R₉, or R₁₀ is hydroxyl, or a pharmaceutically acceptable salt or prodrug thereof; or of Formula IV: 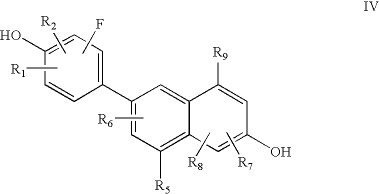
   wherein:
R₁ and R₂ are each, independently, selected from hydrogen, hydroxyl, alkyl of 1-6 carbon atoms, alkenyl of 2-7 carbon atoms, and alkynyl of 2-7 carbon atoms, alkoxy of 1-6 carbon atoms, or halogen;
R₅, R₆, R₇, R₈, or R₉ are each, independently, hydrogen, alkyl of 1-6 carbon atoms, alkenyl of 2-7 carbon atoms, alkynyl of 2-7 carbon atoms, halogen, alkoxy of 1-6 carbon atoms, —CN, —CHO, trifluoromethyl, phenylalkyl of 7-12 carbon atoms, phenyl, or a 5 or 6-membered heterocyclic ring having 1 to 4 heteroatoms selected from O, N or S; wherein the alkyl or alkenyl moieties of R₅, R₆, R₇, R₈, or R₉ may be optionally substituted with hydroxyl, —CN, halogen, trifluoroalkyl, trifluoroalkoxy, —NO₂, or phenyl; wherein the phenyl moiety of R₅, R₆, R₇, R₈, or R₉ may be optionally mono-, di-, or tri-substituted with alkyl of 1-6 carbon atoms, alkenyl of 2-7 carbon atoms, halogen, hydroxyl, alkoxy of 1-6 carbon atoms, —CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;
   wherein at least one of R₅ or R₉ is not hydrogen, or a pharmaceutically acceptable salt or prodrug thereof; or of Formula V: 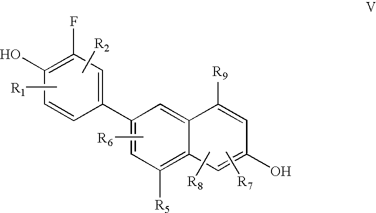
or a pharmaceutically acceptable salt thereof.

In other embodiments, the ERβ selective ligand is of Formula V wherein the 5 or 6-membered heterocyclic ring having 1 to 4 heteroatoms selected from O, N or S is furan, thiophene or pyridine, or a pharmaceutically acceptable salt thereof. In further embodiments, R₅, R₆, R₇, R₈, and R₉ are each, independently, hydrogen, halogen, —CN, alkynyl of 2-7 carbon atoms, alkoxy of 1-6 carbon atoms, —CHO, trifluoromethyl or phenylalkyl of 7-12 carbon atoms, or a pharmaceutically acceptable salt thereof. In still further embodiments, R₆, R₇, and R₈ are hydrogen or a pharmaceutically acceptable salt thereof.

In some embodiments wherein the ERβ selective ligand has the Formula IV, the compound is 7-(4-hydroxyphenyl)-2-naphthol; 7-(3-hydroxyphenyl)-2-naphthol; 6-(4-hydroxyphenyl)-1-naphthol; 6-phenyl-2-naphthol; 6-(3-hydroxyphenyl)-2-naphthol; 6-(3-chlorophenyl)-2-naphthol; 2-fluoro-4-(2-naphthyl)phenol; 6-(3-fluoro-4-hydroxyphenyl)-2-naphthol; 6-(3-chloro-4-hydroxyphenol)-2-naphthol; 1-chloro-6-phenyl-2-naphthol; 1-bromo-6-(4-hydroxyphenyl)-2-naphthol; 1-chloro-6-(4-hydroxyphenyl)-2-naphthol; 1-fluoro-6-(4-hydroxyphenyl)-2-naphthol; 2-hydroxy-6-(4-hydroxyphenyl)-1-naphthonitrile; 6-(4-hydroxyphenyl)-1-phenyl-2-naphthol; 6-(4-hydroxyphenyl)-1-methyl-2-naphthol; 1-chloro-6-(3-fluoro-4-hydroxyphenyl)-2-naphthol; 1-chloro-6-(3-chloro-4-hydroxyphenyl)-2-naphthol; 6-(4-hydroxyphenyl)-1-nitro-2-naphthol; 1-chloro-6-(4-hydroxy-2-methylphenyl)-2-naphthol; 6-(4-hydroxy-2-methylphenyl)-2-naphthol; 6-(4-hydroxy-2-methoxyphenyl)-2-naphthol; 6-(2-chloro-4-hydroxyphenyl)-2-naphthol; 1-chloro-6-(2-chloro-4-hydroxyphenyl)-2-naphthol; 6-(2-fluoro-4-hydroxyphenyl)-2-naphthol; 6-(2,5-difluoro-4-hydroxyphenyl)-2-naphthol; 6-(2,6-difluoro-4-hydroxyphenyl)-2-naphthol; 1-chloro-6-(2-fluoro-4-hydroxyphenyl)-2-naphthol; 1-chloro-6-(2,5-difluoro-4-hydroxyphenyl)-2-naphthol; 1-chloro-6-(2,6-difluoro-4-hydroxyphenyl)-2-naphthol; 8-fluoro-6-(4-hydroxyphenyl)-2-naphthol; 1-chloro-8-fluoro-6-(4-hydroxyphenyl)-2-naphthol; 8-chloro-6-(4-hydroxyphenyl)-2-naphthol; 1,5-dichloro-8-fluoro-6-(4-hydroxyphenyl)-2-naphthol; 2-chloro-4-(2-naphthyl)phenol; 3-bromo-8-chloro-6-(4-hydroxyphenyl)-2-naphthol; 1,8-dichloro-6-(4-hydroxyphenyl)-2-naphthol; 3-bromo-1,8-dichloro-6-(4-hydroxyphenyl)-2-naphthol; 7-hydroxy-3-(4-hydroxyphenyl)-1-naphthonitrile; 8-chloro-3-(4-hydroxyphenyl)-7-hydroxy-1-naphthonitrile; 8-chloro-3-(3-fluoro-4-hydroxyphenyl)-7-hydroxy-1-naphthonitrile; 6-(3,5-difluoro-4-hydroxyphenyl)-2-naphthol; 1-chloro-6-(3,5-difluoro-4-hydroxyphenyl)-2-naphthol; 8-bromo-7-hydroxy-3-(4-hydroxyphenyl)-1-naphthonitrile; 8-fluoro-6-(3-fluoro-4-hydroxyphenyl)-2-naphthol; 1-chloro-8-fluoro-6-(3-fluoro-4-hydroxyphenyl)-2-naphthol; 3-(3-fluoro-4-hydroxyphenyl)-7-hydroxy-1-naphthonitrile; 3-(3,5-difluoro-4-hydroxyphenyl)-7-hydroxy-1-naphthonitrile; or a pharmaceutically acceptable salt or prodrug thereof.

The preparation of ERβ selective ligands having Formulae III, IV and V are described in U.S. Pat. No. 6,914,074 and patent application, WO 03/051805, incorporated herein by reference in their entireties.

In further embodiments of the methods of the present invention, the ERβ selective ligand is of Formula VI: 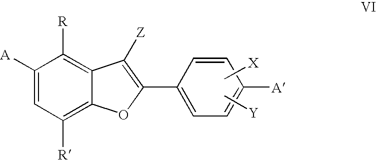
wherein:
A is alkyl of 1-6 carbon atoms, halogen, trifluoroalkyl of 1-6 carbon atoms, hydroxyalkyl of 1-6 carbon atoms, —CO₂H, —NH₂, or —OP;
A′ is —OP, —CO₂P, halogen, or hydroxyalkyl;
P is hydrogen, alkyl of 1-6 carbon atoms, or phenyl;
Z is hydrogen, alkyl of 1-6 carbon atoms, halogen, —NO₂, —CN, trifluoroalkyl of 1-6 carbon atoms, —COP, —CO₂P, or —C(P)═N—OP;
R and R′ are each, independently, hydrogen, alkyl of 1-6 carbon atoms, alkenyl of 2-7 carbon atoms, halogen, —OP, —SP, —SOP, —SO₂P, —SCN, trifluoroalkyl of 1-6 carbon atoms, —CF₂CF₃, trifluoroalkoxy of 1-6 carbon atoms, —NO₂, —NH₂, —NHOP, hydroxyalkyl of 1-6 carbon atoms, alkoxyalkyl of 1-6 carbon atoms per alkyl group, -alkyl-SP, -alkyl-SOP, -alkyl-SO₂P, —CN, -alkyl-CN, -alkenyl-CN, -alkylSCN, —CHFCN, —CF₂CN, -alkenyl-NO₂, haloalkyl of 1-6 carbon atoms, dihaloalkenyl of 2-7 carbon atoms, —COP, —COCF₃, —CO₂P, —CONR₁R₂, -alkyl-CONR, R₂, -alkenyl-CONR₁R₂, -alkyl-COP, -alkenyl-COP, -alkenyl-CO₂P, -alkenyl-CO₂P, oxadiazolyl, furyl, thienyl, pyrrolyl, imidazolyl, triazolyl, or tetrazolyl;
X and Y are each, independently, hydrogen, alkyl of 1-6 carbon atoms, halogen, —NO₂, —CN, trifluoroalkyl of 1-6 carbon atoms, —OP, hydroxyalkyl of 1-6 carbon atoms, —CO₂H, or phenyl which is optionally mono- or di-substituted with hydroxyl, benzyloxy, alkoxy of 1-6 carbon atoms, or —OCH₂CH₂NR₁R₂;
R₁ and R₂ are each, independently, hydrogen, alkyl of 1-6 carbon atoms, or alkoxy of 1-6 carbon atoms; or R₁ and R₂ are concatenated together as —(CH₂)ₚ—;
p=2-6;
or a pharmaceutically acceptable salt or prodrug thereof.

The preparation of ERβ selective ligands having Formula VI are described in U.S. Pat. No. 6,774,248 and patent application, WO 03/051860, incorporated herein by reference in their entireties.

In further embodiments of the present invention, the ERβ selective ligand is of Formula VII: 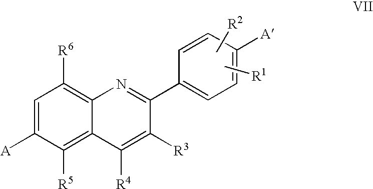
wherein:
A and A′ are each, independently, OH or OP;
P is alkyl, alkenyl, benzyl, acyl, aroyl, alkoxycarbonyl, sulfonyl or phosphoryl;
R¹ and R² are each, independently, H, halogen, C₁-C₆ alkyl, C₂-C₇ alkenyl, or C₁-C₆ alkoxy;
R³ is H, halogen, or C₁-C₆ alkyl;
R⁴ is H, halogen, C₁-C₆ alkyl, C₂-C₇ alkenyl, C₂-C₇ alkynyl, C₃-C₇ cycloalkyl, C₁-C₆ alkoxy, —CN, —CHO, acyl (i.e., alkylcarbonyl such as acetyl), or heteroaryl, e.g., where heteroaryl is an aromatic ring of up to 5 carbon atoms and at least one heteroatom selected from O, N or S including furyl, thienyl, pyrrolyl, pyridyl, pyrimidyl, oxazolyl, thiazolyl, etc.;
R⁵ and R⁶ are each, independently, H, halogen, C₁-C₆ alkyl, C₂-C₇ alkenyl, C₂-C₇ alkynyl, C₃-C₇ cycloalkyl, C₁-C₆ alkoxy, —CN, —CHO, acyl, phenyl, aryl or heteroaryl, provided that at least one of R⁴, R⁵ and R⁶ is halogen, C₁-C₆ alkyl, C₂-C₇ alkenyl, C₂-C₇ alkynyl, C₃-C₇ cycloalkyl, C₁-C₆ alkoxy, —CN, —CHO, acyl, phenyl, aryl or heteroaryl;
wherein the alkyl or alkenyl moieties of R⁴, R⁵ or R⁶ may be optionally substituted with halogen, OH, —CN, trifluoroalkyl, trifluoroalkoxy, —NO₂, or phenyl;
wherein the alkynyl moiety of R⁴, R⁵ or R⁶ may be optionally substituted with halogen, —CN, —CHO, acyl, trifluoroalkyl, trialkylsilyl, or optionally substituted phenyl;
wherein the phenyl moiety of R⁵ or R⁶ may be optionally mono-, di-, or tri-substituted with halogen, C₁-C₆ alkyl, C₂-C₇ alkenyl, OH, C₁-C₆ alkoxy, —CN, —CHO, —NO₂,
amino, C₁-C₆ alkylamino, di-(C₁-C₆)alkylamino, thiol, or C₁-C₆ alkylthio;
provided that when each of R⁴, R⁵ and R⁶ are H, C₁-C₆ alkyl, C₂-C₇ alkenyl, or C₁-C₆ alkoxy, then at least one of R¹ and R² is halogen, C₁-C₆ alkyl, C₂-C₇ alkenyl, or C₁-C₆ alkoxy;
provided that at least one of R⁴ and R⁶ is other than H;
or a N-oxide thereof;
or Formula VIII: 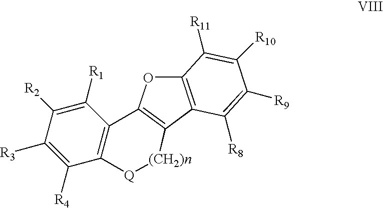
   wherein:
Q has the structure i, ii or iii: 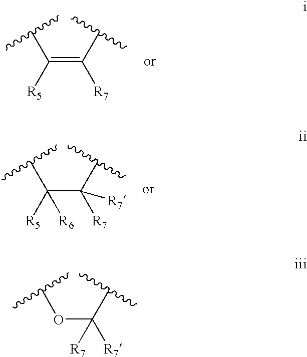
R₁, R₄, R₅, R₆ R₇, R_{7′}, R₈ and R₁₁ are each independently selected from the group consisting of hydrogen, C₁-C₆ alkyl, —OR₂₀, halogen, —CF₃, —CF₂CF₃, —CH₂CF₃, —SR₂₀, NR₂₀R₂₁, —CN, —CH₂CN, —CH₂CH₂CN, —CH═CHCN, —NO₂, —CH₂NO₂, —CH₂CH₂NO₂, —CH═CHNO₂ and —COR₂₀;
n=0 or 1;
each R₂₀ and R₂₁ is independently selected from the group consisting of hydrogen, C₁-C₆ alkyl, —CF₃, benzyl, —CO₂(C₁-C₆ alkyl) and —CO(C₁-C₆ alkyl);
provided that:
   a) one of R₂ or R₃ must be —OR₂₀;
   b) one of R₉ or R₁₀ must be —OR₂₀;
   c) when R₂ is —OR₂₀, then R₁ and R₃ are selected independently from the group consisting of hydrogen, halogen, C₁-C₆ alkyl, —CF₃, —CF₂CF₃, —CH₂CF₃, —SR₂₀, —CN, —CH₂CN, —CH₂CH₂CN, —CH═CHCN, —NO₂, —CH₂NO₂, —CH₂CH₂NO₂, —CH═CHNO₂ and —COR₂₀;
   d) when R₃ is —OR₂₀, then R₂ and R₄ are selected independently from the group consisting of hydrogen, C₁-C₆ alkyl, halogen, —CF₃, —CF₂CF₃, —CH₂CF₃, —SR₂₀, —CN, —CH₂CN, —CH₂CH₂CN, —CH═CHCN, —NO₂, —CH₂NO₂, —CH₂CH₂NO₂, —CH═CHNO₂ and —COR₂₀;
   e) when R₉ is —OR₂₀, then R₈ and R₁₀ are selected independently from the group consisting of hydrogen, C₁-C₆ alkyl, halogen, —CF₃, —CF₂CF₃, —CH₂CF₃, —SR₂₀, —CN, —CH₂CN, —CH₂CH₂CN, —CH═CHCN, —NO₂, —CH₂NO₂, —CH₂CH₂NO₂, —CH═CHNO₂ and —COR₂₀;
   f) when R₁₀ is —OR₂₀, then R₉ and R₁₁ are selected independently from the group consisting of hydrogen, C₁-C₆ alkyl, halogen, —CF₃, —CF₂CF₃, —CH₂CF₃, —SR₂₀, —CN, —CH₂CN, —CH₂CH₂CN, —CH═CHCN, —NO₂, —CH₂NO₂, —CH₂CH₂NO₂, —CH═CHNO₂ and —COR₂₀; and
   g) when Q has the structure iii, and R₇, R₇, R₈, R₉, R₁₁ are each H, and n=0, then R₁₀ is not OR₂₀,
or a pharmaceutically acceptable salt or prodrug thereof;
or Formula IX: 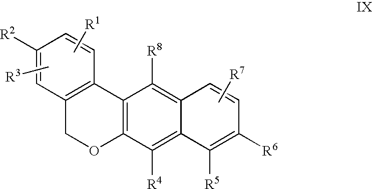
   wherein:
R₁, R₂, R₃, R₅, R₆, R₇, and R₈ are each, independently, selected from hydrogen, hydroxyl, C₁-C₆ alkyl, C₁-C₆alkoxy, or halogen;
R₄ is hydrogen, C₁-C₆ alkyl, halogen, C₁-C₆ alkoxy, —CN, C₂-C₈ alkenyl, —CHO, aryl, furyl, thienyl, pyrimidinyl, or pyridinyl;
provided that at least one of R₁-R₈ is other than H; (wherein “Aryl,” as used above as a group or part of a group, refers to an optionally substituted aromatic 5- to 13-membered mono- or bi- carbocyclic ring such as phenyl or naphthyl; and in some embodiments, phenyl moieties are optionally substituted with C₁-C₆ alkyl, C₂-C₇ alkenyl, halogen, hydroxyl, C₁-C₆ alkoxy, —CN, —NO₂, amino, C₁-C₆ alkylamino, dialkylamino of 1-6 carbon atoms per alkyl group, thio, C₁-C₆ alkylthio, C₁-C₆ alkylsulfinyl, C₁-C₆ alkylsulfonyl, C₂-C₇ alkoxycarbonyl, of 2-7 carbon atoms, alkylcarbonyl of 2-7 carbon atoms, trifluoroalkxoy, benzylnitrile or benzoyl);
or a pharmaceutically acceptable salt or prodrug thereof;
or of Formula X: 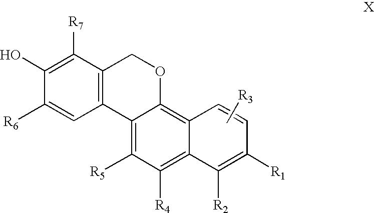
   wherein:
R₁ and R₂ are each, independently, selected from hydrogen, hydroxyl, alkyl of 1-6 carbon atoms, alkenyl of 2-6 carbon atoms, alkynyl of 2-7 carbon atoms, alkoxy of 1-6 carbon atoms, or halogen; wherein the alkyl or alkenyl moieties of R₁ or R₂ may be optionally substituted with hydroxyl, —CN, halogen, trifluoroalkyl, trifluoroalkoxy, —NO₂, or phenyl; and provided that at least one of R₁ or R₂ is hydroxyl;
R₃, R₄, R₅, R₆, and R₇ are each, independently, hydrogen, alkyl of 1-6 carbon atoms, halogen, alkoxy of 1-6 carbon atoms, —CN, alkenyl of 2-7 carbon atoms, alkynyl of 2-7 carbon atoms, —CHO, phenyl, or a 5 or 6-membered heterocyclic ring having 1 to 4 heteroatoms selected from O, N or S; wherein the alkyl or alkenyl moieties of R₄, R₅, R₆, or R₇ may be optionally substituted with hydroxyl, —CN, halogen, trifluoroalkyl, trifluoroalkoxy, —NO₂, or phenyl;

wherein the phenyl moiety of R₄ or R₅ may be optionally mono-, di-, or tri-substituted with alkyl of 1-6 carbon atoms, alkenyl of 2-7 carbon atoms, halogen, hydroxyl, alkoxy of 1-6 carbon atoms, —CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;
or a pharmaceutically acceptable salt or prodrug thereof.

The preparation of ERβ selective ligands having Formula VII is described in U.S. patent application Ser. No. 10/846,216 and PCT application, WO 04/103973. The preparation of ERβ selective ligands having Formula VIII is described in U.S. Patent Application Ser. No. 60/584,516 filed Jul. 1, 2004. The preparation of ERβ selective ligands having Formula IX is disclosed in U.S. Patent Application Ser. No. 60/547,967 and PCT application, WO 05/082880. The preparation of ERβ selective ligands having Formula X is disclosed in U.S. Pat. No. 6,723,747 and European Patent No. EP 1453820 B1. Each of the foregoing patents and applications is incorporated herein by reference in its entirety.

In some embodiments of the methods disclosed herein, the ERβ selective ligand is substantially free of ERβ antagonist activity.

In further embodiments, the methods disclosed herein are used to treat Parkinson's disease. In some embodiments, the present invention provides methods for ameliorating a symptom of Parkinson's disease. Examples of such symptoms include but are not limited to poor balance, Parkinsonian gait, bradykinesia, rigidity, tremor, speech changes, loss of facial expression, micrographia, difficulty swallowing, drooling, pain, dementia or confusion, sleep disturbances, constipation, skin problems, depression, fear, anxiety, memory difficulties, slowed thinking, sexual dysfunction, urinary problems, fatigue, aching, and loss of energy.

In a further aspect, the present invention provides methods for the amelioration of a symptom of a cognitive disease or disorder. In some such embodiments, the disease or disorder is schizophrenia, multiple sclerosis, depression, stroke, Alzheimer's disease or anxiety. In some such embodiments, a patient is identified as having a symptom of the cognitive disease or disorder, and is administered a therapeutically effective amount of an ERβ selective ligand, or a pharmaceutically acceptable salt or prodrug thereof, wherein the ERβ selective ligand is substantially free of ERβ antagonist activity. In some embodiments, the ERβ selective ligand has one of the formulas I-X as described above.

In some embodiments, the invention provides methods for ameliorating a symptom of schizophrenia. In some such embodiments, the symptoms of schizophrenia being treated can be positive symptoms, negative symptoms and/or cognitive symptoms. Examples of positive symptoms of schizophrenia include, but are not limited to, hallucinations, delusions and/or paranoia. Examples of negative symptoms of schizophrenia include, but are not limited to, social withdrawal, flat affect, anhedonia and/or decreased motivation. In still further embodiments of the methods of the invention, the symptom of schizophrenia is a cognitive symptom. Examples of such cognitive symptoms include, but are not limited to, severe deficit in attention, object naming, working memory, long-term memory storage or executive functioning, a slowing of information processing or neural activity, or long term depression.

In some embodiments, the invention provides methods for ameliorating a symptom of multiple sclerosis. Examples of such symptoms include, but are not limited to, optic neuritis blurred vision, eye pain, loss of color vision, blindness, diplopia double vision, nystagmus jerky eye movements, ocular dysmetria constant under- or over-shooting eye movements, internuclear ophthalmoplegia, nystagmus, diplopia, movement and sound phosphenes, nystagmus, diplopia, afferent pupillary defect, motor paresis, monoparesis, paraparesis, hemiparesis, quadraparesis plegia, paraplegia, hemiplegia, tetraplegia, quadraplegia, spasticity, dysarthria, muscle atrophy, spasms, cramps, hypotonia, clonus, myoclonus, myokymia, restless leg syndrome, footdrop dysfunctional reflexes (babinski's, hoffman's, chaddock's), paraesthesia, anaesthesia, neuralgia, neuropathic and neurogenic pain, I'hermitte's, proprioceptive dysfunction, trigeminal neuralgia, ataxia, intention tremor, dysmetria, vestibular ataxia, vertigo, speech ataxia, dystonia, dysdiadochokinesia, frequent micturation, bladder spasticity, flaccid bladder, detrusor-sphincter dyssynergia, erectile dysfunction, anorgasmy, retrograde ejaculation, frigidity, constipation, fecal urgency, depression, cognitive dysfunction, dementia, mood swings, emotional lability, euphoria, bipolar syndrome, anxiety, aphasia, dysphasia, fatigue, uhthoff's symptom, gastroesophageal reflux and/or sleeping disorders.

In some embodiments, the present invention provides methods for ameliorating a symptom of depression. Examples of such symptoms include, but are not limited to, depressed feeling or mood, loss of interest or pleasure in some or all activities, changes in appetite, weight or sleep patterns, lack of energy, fatigue, low self esteem, diminished capacity for thinking, concentration, or decisiveness, feelings of hopelessness or worthlessness, psychomotor agitation or retardation, self-reproach, inappropriate guilt, frequent thoughts of death or suicide, plans and/or attempts to commit suicide.

In some embodiments, the present invention provides methods for ameliorating a symptom of Alzheimer's disease. Examples of such symptoms include, but are not limited to, impairment in memory, attention, judgment, decision-making, orientation to physical surroundings, language, speed-dependent activities, abstract reasoning, visuospatial abilities, executive functioning, and behavioral disturbances, disinterest and passivity, apathy, inappropriate dressing, poor self care, agitation, violent outbursts, aggression, depression, anxiety, hallucinations, delusions, changes in personality and mood changes, and dementia.

In some embodiments, the present invention provides methods for ameliorating a symptom of anxiety. Examples of such symptoms include, but are not limited to, feelings of apprehension and fear, which are accompanied by physical symptoms that may reflect a category of anxiety disorder. For example, symptoms of Generalized Anxiety Disorder (GAD) include, e.g., trembling, muscle aches, insomnia, abdominal upsets, dizziness and irritability. Obsessive-Compulsive Disorder (OCD) is symptomized by, e.g., persistent, recurring thoughts (obsessions), which may lead the individual to perform ritual or routine behavior (compulsions). Panic Disorder symptoms include, e.g., heart palpitations, chest pain, chest discomfort, sweating, trembling, tingling sensations, feeling of choking, fear of losing control, fear of dying, and feelings of unreality. Three main symptoms are associated with Post-Traumatic Stress Disorder (PTSD), which are (1) “reliving” the traumatic event, such as flashbacks, nightmares, intrusive thoughts and recollections, (2) avoidance behaviors and emotional numbing, and (3) hypersensitivity such as an inability to sleep, anxious feelings, overactive startle response, hypervigilance, irritability and outbursts of anger. Physical symptoms of Social Anxiety Disorder include, e.g., heart palpitations, faintness, blushing and profuse sweating.

In some embodiments, the present invention provides methods for ameliorating a symptom of stroke. Examples of traditional symptoms include, e.g., hemiparesis, vertigo, numbness, aphasia, dysarthria, dysphasia, facial drooping, loss of balance or coordination, inability to walk, changes in sensation and vision problems. Nontraditional symptoms include, e.g., headache, facial pain, limb pain, disorientation and change in consciousness, chest pain, shortness of breath, palpitations and neurologic symptoms such as hiccups, nausea and general weakness.

In some embodiments of each of the foregoing, the methods comprise identifying a patient suffering from a symptom of the disease or disorder, and administering a therapeutically effective amount of an ERβ selective ligand, or a pharmaceutically acceptable salt or prodrug thereof, wherein the ERβ selective ligand is substantially free of ERβ antagonist activity.

In some embodiments, an ERβ selective ligand which is substantially free of ERβ antagonist activity is used in the preparation of a medicament for treating Parkinson's disease in a patient identified as having said disease. In some embodiements, an ERβ selective ligand which is substantially free of ERβ antagonist activity is used in the preparation of a medicament for ameliorating a symptom of Parkinson's disease in a patient identified as having said disease and having said symptom thereof. In some embodiments, an ERβ selective ligand which is substantially free of ERβ antagonist activity is used in the preparation of a medicament for ameliorating a symptom of a cognitive disease or disorder in a patient identified as having said disease and having said symptom thereof; wherein said disease or disorder is selected from multiple sclerosis, depression, schizophrenia, stroke, Alzheimer's disease or anxiety.

As used herein, the term “substantially free of antagonist activity” means that the ERβ selective ligand when co-administered with estradiol has at least greater than or equal to 65 percent, preferably at least about >70 percent, more preferably at least about >80 percent, and most preferably at least >90 percent the activity seen when estradiol is administered alone as determined by a cell-based transcriptional assay (Harris H et al., 2001 Endocrinology 142(2): 645-652, Yang C et al., 2004 Bioorganic & Medicinal Chemistry 12:2553-2570) or that helix 12 of the ERβ selective ligand is in the closed agonist confirmation as determined by an x-ray co-crystal of the compound with ERβ ligand binding domain (Malamas M S et al., 2004 J. Med. Chem. 47(21): 5021-5040).

Pharmaceutically acceptable salts can be formed from organic and inorganic acids, for example, acetic, propionic, lactic, citric, tartaric, succinic, fumaric, maleic, malonic, mandelic, malic, phthalic, hydrochloric, hydrobromic, phosphoric, nitric, sulfuric, methanesulfonic, naphthalenesulfonic, benzenesulfonic, toluenesulfonic, camphorsulfonic, and similarly known acceptable aids when a compound of this invention contains a basic moiety. Salts may also be formed from organic and inorganic bases, such as alkali metal salts (for example, sodium, lithium, or potassium) alkaline earth metal salts, ammonium salts, alkylammonium salts containing 1-6 carbon atoms or dialkylammonium salts containing 1-6 carbon atoms in each alkyl group, and trialkylammonium salts containing 1-6 carbon atoms in each alkyl group, when a compound of this invention contains an acidic moiety.

The terms alkyl, alkenyl, and alkynyl include both branched and straight chain moieties. Examples include methyl, ethyl, propyl, butyl, isopropyl, sec-butyl, tert-butyl, vinyl, allyl, acetylene, 1-methyl vinyl, and the like. When alkyl or alkenyl moieties are substituted, they may typically be mono-, di-, tri- or persubstituted. Examples for a halogen substituent include 1-bromo vinyl, 1-fluoro vinyl, 1,2-difluoro vinyl, 2,2-difluorovinyl, 1,2,2-trifluorovinyl, 1,2-dibromo ethane, 1,2 difluoro ethane, 1-fluoro-2-bromo ethane, CF₂CF₃, CF₂CF₂CF₃, and the like. The term halogen includes bromine, chlorine, fluorine, and iodine. The term aryl means phenyl, 1-naphthyl, or 2-naphthyl. Preferred 5-6 membered heterocyclic rings include furan, thiophene, pyrrole, isopyrrole, pyrazole, imidazole, triazole, dithiole, oxathiole, isoxazole, oxazole, thiazole, isothiazolem oxadiazole, furazan, oxatriazole, dioxazole, oxathiazole, tetrazole, pyran, pyridine, pyridazine, pyrimidine, pyrazine, triazine, oxazine, oxathiazine, or oxadiazine. It is more preferred that the heterocyclic ring is furan, thiophene, or thiazole.

As used in accordance with this invention, the term “treatment” or “treating” means curing, ameliorating or reversing the progress of a disease or disorder, or ameliorating or reversing one or more symptoms or side effects of such disease or disorder.

As used in accordance with this invention, the term “administering” means either directly administering the ERβ selective agonists, or administering a prodrug, derivative, or analog of the ERβ selective agonist that will form an effective amount of the ERβ selective agonist within the CNS.

As used in accordance with this invention, the term “ERβ selective ligand” means that the binding affinity (as measured by IC₅₀, where the IC₅₀ of 17β-estradiol is not more than 3 fold different between ERα and ERβ) of the ligand to ERβ is at least about 10 times greater than its binding affinity to ERα in a standard pharmacological test procedure that measures the binding affinities to ERα and ERβ. It is preferred that the ERβ selective ligand will have a binding affinity to ERβ that is at least about 20 times greater than its binding affinity to ERα. It is more preferred that the ERβ selective ligand will have a binding affinity to ERβ that is at least about 50 times greater than its binding affinity to ERα.

When administered for the treatment or inhibition of a particular disease state or disorder, it is understood that the effective dosage may vary depending upon the particular ERβ agonist utilized, the mode of administration, the condition being treated, and severity thereof, as well as the various physical factors related to the individual being treated. Effective administration of the ERβ selective ligand of this invention may be in any of a variety of dosage regimes such as single dosage, multiple dosage, and delay or time release dosage forms. The projected daily dosages are expected to vary with route of administration. The selection of the appropriate administration and dosage forms for an individual patient will be apparent to those skilled in the art.

Such doses may be administered in any manner useful in directing the active ERβ agonists herein to the recipient's bloodstream, including orally, via implants, parentally (including intravenous, intraperitoneal, intraarticularly and subcutaneous injections), rectally, intranasally, topically, ocularly (via eye drops), vaginally, and transdermally.

Oral formulations containing the active ERβ agonists of this invention may comprise any conventionally used oral forms, including tablets, capsules, buccal forms, troches, lozenges and oral liquids, suspensions or solutions. Capsules may contain mixtures of the active compound(s) with inert fillers and/or diluents such as the pharmaceutically acceptable starches (e.g., corn, potato or tapioca starch), sugars, artificial sweetening agents, powdered celluloses, such as crystalline and microcrystalline celluloses, flours, gelatins, gums, etc. Useful tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Preferred surface modifying agents include nonionic and anionic surface modifying agents. Representative examples of surface modifying agents include, but are not limited to, poloxamer 188, benzalkonium chloride, calcium stearate, cetostearyl alcohol, cetomacrogol emulsifying wax, sorbitan esters, colloidol silicon dioxide, phosphates, sodium dodecylsulfate, magnesium aluminum silicate, and triethanolamine. Oral formulations herein may utilize standard delay or time release formulations to alter the absorption of the active compound(s). The oral formulation may also consist of administering the active ingredient in water or a fruit juice, containing appropriate solubilizers or emulsifiers as needed.

In some cases it may be desirable to administer the compounds directly to the airways in the form of an aerosol.

The compounds of this invention may also be administered parenterally (i.e., subcutaneously, intravenously, intramuscularly) or intraperitoneally. Solutions or suspensions of these active ERβ agonists as a free base or pharmacologically acceptable salt can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparations contain a preservative to inhibit the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

For the purposes of this disclosure, transdermal administrations are understood to include all administrations across the surface of the body and the inner linings of bodily passages including epithelial and mucosal tissues. Such administrations may be carried out using the present compounds, or pharmaceutically acceptable salts thereof, in lotions, creams, foams, patches, suspensions, solutions, and suppositories (rectal and vaginal).

Transdermal administration may be accomplished through the use of a transdermal patch containing the active compound and a carrier that is inert to the active compound, is non-toxic to the skin, and allows delivery of the agent for systemic absorption into the blood stream via the skin. The carrier may take any number of forms such as creams and ointments, pastes, gels, and occlusive devices. The creams and ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the active ERβ agonist into the blood stream such as a semi-permeable membrane covering a reservoir containing the active ingredient with or without a carrier, or a matrix containing the active ingredient. Other occlusive devices are known in the literature.

Suppository formulations may be made from traditional materials, including cocoa butter, with or without the addition of waxes to alter the suppository's melting point, and glycerin. Water soluble suppository bases, such as polyethylene glycols of various molecular weights, may also be used.

Response by patients with schizophrenia, Parkinson disease, multiple sclerosis, cognitive deficiencies and other brain, memory, learning and cognitive disorders can generally be determined by standard test procedures within the skill of those in the art.

The following examples are merely illustrative of the present invention and should not be considered limiting of the scope of the invention in any way. These examples and equivalents thereof will become more apparent to those skilled in the art in light of the present disclosure and the accompanying claims.

### EXAMPLES

Patients can be evaluated for cognitive diseases or disorders by any of the tests known in the art. Preclinically, animals can be evaluated for blockade/attenuation of symptoms associated with schizophrenia. Positive symptoms in animal models of schizophrenia can be evaluated by measuring changes in the overall level of activity of dopamine (DA) activity with concomitant parallel changes in locomotor activity (Depoortere R et al., 2003 Neuropsychopharmacology 28(11):1889-902), D-amphetamine (AMPH) and phencyclidine (PCP) via induction of model psychosis or locomotor hyperactivity (Freed W J et al., 1984 Neuropharmacology 23(2A):175-81; Sams-Dodd, F. 1998 Neuropsychopharmacology 19(1): 18-25). For example, Depoortere et al. have described tests for evaluating locomotor activity, catalepsy, climbing and stereotypy, which relate to positive symptomology and side effect profile, by characterizing compounds with typical and atypical antipsychotic efficacy (2003). Attenuation in apomorphine-induced climbing, stereotypy and catalepsy (AIC) can be evaluated as described by Fung Y K et al. 1986 Pharmacol Biochem Behav. 1986 24(1):139-41 and Fung, et aL, 1987 Steroids 49(4-5):287-94. Additionally, negative symptoms of schizophrenia can be evaluated by measuring social interaction under the influence of NMDA antagonists such as PCP (Sams-Dodd F 1998).

Cognitive symptoms of memory, including those from Alzheimer's disease and stroke, can be evaluated by such models as the Fear Conditioning Paradigm (Gould T J et al., 2002 Behav Pharmacol. 13(4):287-94; Hamm A O et al., 2003 Brain 126(Pt 2):267-75) and Radial Arm Test (Aggleton J P et al., 1996 Behav Brain Res. 19(2):133-46), while spatial reference memory and learning can be evaluated in the Morris watermaze (Bontempi B et al., 1996 Eur J Neurosci. 8(11):2348-60). Additionally, memory and hippocampal hypo-functioning can be assessed by measuring the restoration of synaptic plasticity in ovariectomized (OVX) female rats. (Day and Good, 2005 Jan., Neurobiol Learn Mem., 83(1): 13-21). Further, changes in attention function because of schizophrenia can be examined by the five (5) Choice Serial Reaction Time Test (5CSRT) (see Muir J L, et al., 1995 Psychopharmacology (Berl) 118(1): 82-92; Robbins et al., 1998 Ann N Y Acad Sci. 846:222-37).

Further for stroke, the Tamura model is one of the best-characterized focal ischemia models whereby the middle cerebral artery is occluded by electro-coagulation. Also the Johnson and McCarty model, the spontaneously hypertensive rat (SHR), and the newer endothelin-1 model may be used for evaluating stroke (Johnson M P, McCarty D R et al., 1998 Life Sci. 63(4):241-53; Sharkey J and Butcher S P 1995 J Neurosci Methods 60(1-2):125-31). In the examination of ERβ agonists for stroke, the following models may be use: (1) MCOA using stereotaxic infusion of Et-1, (2) horizontal and inclined balance beam to assess sensorimotor performance after Et-1 MCAO (Petullo D et al., 1999 Life Sci 64(13): 1099-108; Lecci A et al., 1990 Neuropeptides 16(1): 21-4) (3) staircase test to measure skilled paw use after Et-1 MCAO (Marston H M et al., 1995 Neuroreport 6(7):1067-71), (4) Tamura model of MCAO to test neuroprotective agents, and (5) spontaneously hypertensive rat model of MCAO to test neuroprotective agents (Dawson D A and D Martin et al., 1996 Neurosci Lett 218(1):41-4; Ohtani K H et al., 2003 Neurochem Int 42(5):375-84).

An assessment of depression can be measured using the learned helplessness model (Haracz J L et al., 1988 Biol Psychiatry 23(4):388-96; Shors T J and Leuner B 2003 J Affect Disord 74(1):85-96) and the forced swim test (Walf A A et al., 2002 Pharmacol Biochem Behav 78(3):523-9). Depression and anxiety can both be evaluated by tail suspension-induced disuse atrophy in ovariectomized rats (Ohmori S et al., 2001 Environ Med 45(1):12-4). Further, anxiety may be assessed by the following tests: (1) the Geller-Seifter conflict test (Babbini M et al., 1982 Pharmacol Biochem Behav 17(1): 43-8; Shimizu H et al., 1992 Jpn J Pharmacol 58(3): 283-9), (2) social interaction (Gonzalez L E et al., 1998 Pharmacol Biochem Behav 59(4): 787-92), (3) light/dark exploration (Holmes A et al., 2001 Behav Brain Res 122(2): 159-67), (4) elevated plus-maze (Andreatini R and L F Bacellar 1999 Braz J Med Biol Res 32(9): 1121-6), (5) defensive burying (Overmier J B et al., 1994 Biol Psychiatry 36(10): 703-4), and (6) the thirsty rat conflict (Mendelson W B et al., 1983 Life Sci 32(19): 2241-6; Overton D A et al., 1993 Psychopharmacology (Berl) 112(2-3): 270-6).

Parkinson's disease can be assessed by measuring the neurotoxicity of MPTP in rats (Lee E H et al., 1992 Chin J Physiol 35(4):317-36). Also experimentally induced striatal DA depletion in animals is a valid model of Parkinsonism (Schultz W 1982 Prog Neurobiol 18(2-3): 121-66). The capacity of certain substances to damage catecholaminergic neurons has been used extensively to produce DA deficiency in animals (Annett L E et al., 1994 Exp Neurol 125(2): 228-46).

Multiple sclerosis can be evaluated by the experimental autoimmune encephalomyelitis (EAE) model (Liu H Y et al., 2002 J Neurosci Res 70(2): 238-48). Each of the foregoing publications are incorporated herein by reference in their entirety.

### EXAMPLE 1

### Evaluation of Positive Symptomology of Schizophrenia: Pharmacologically Induced Locomotor Activity (LMA), Catalepsy, Apomorphine Induced Climbing (AIC) and Stereotypy

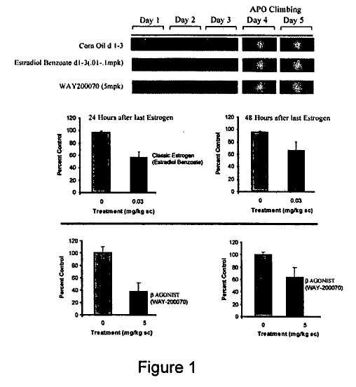

Male C56/BL6 mice were pretreated with estradiol benzoate 0.1, 0.3 and 1 mg/kg and an estrogen beta agonist, 7-bromo-2-(4-hydroxyphenyl)-1,3-benzoxazol-5-ol for three (3) consecutive days, and then evaluated for locomotor activity, catalepsy, AIC and stereotypy. Estradiol benzoate attenuated AIC at 24 and 48 hours (approximately 55%), while the estrogen beta agonist, 7-bromo-2-(4-hydroxyphenyl)-1,3-benzoxazol-5-ol, outperformed estradiol benzoate by inducing a sixty percent (60%) blockade of AIC in the male mice. Results of the study are shown in FIG. 1. It can be seen that ERβ agonists effectively treat the pharmaceutically induced positive symptoms associated with schizophrenia.

### EXAMPLE 2

### Evaluation of the Estrogen Beta Female Knock Out (BERKO) Mice on Phencyclidine (PCP) Locomotor Activity (LMA)

Animals were treated for five (5) days with PCP. Following this period, the animals were given a 4 day withdrawal period. One group received 0.3mg/kg estradiol benzoate on day 5. All subjects were then given a sub-effective dose of PCP that has been shown to increase locomotor activity during PCP withdrawal. In this study it was found that estradiol benzoate at the 0.3mg/kg dose successfully blocked the effect of PCP induced LMA in the βERKO female mice. Thus, the classic estrogen agonist, estradiol benzoate, effectively blocks the effects of PCP on LMA, with other ERβ agonists likely to behave similarly.

### EXAMPLE 3

### Evaluation of the Estrogen Beta Female Knock Out (BERKO) Mice on Contextual Fear Conditioning

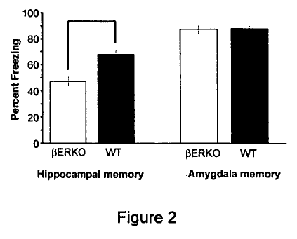

Using basic Pavlovian conditioning, rodents female βERKO knockout and wildtype mice were exposed to an operant chamber (context) and received a 0.5 mA shock (Gould T J, McCarty M M et al., 2002 Behav Pharmacol. 13(4):287-94). The rodents readily learned that the shock is predicted on context, such that when they are placed back in the operant chamber at a later date, they show the fear response that was originally observed in the presence of the shock. As shown in FIG. 2, it was found that the βERKO mice had a deficit for hippocampal, but not amygdala, dependent memory.

### EXAMPLE 4

### Evaluation of Working Memory by the Spatial Reference Memory (Radial Arm Maze) Test

Female rats were habituated to a water deprivation schedule and to the radial maze for at least one week prior to acquisition. The rats were ovariectomized six to eight (6-8) weeks before testing. Subjects were then treated with either estradiol benzoate (0.02 mg/kg), the ERfl agonist, 7-bromo-2-(4-hydroxyphenyl)-1,3-benzoxazol-5-ol, or an ERα agonist. Estradiol benzoate was administered in oil s.c for two (2) days, while the 7-bromo-2-(4-hydroxyphenyl)-1,3-benzoxazol-5-ol and the ERα agonist were administered for six (6) days at 10 mg/kg. Then the rats were run through the acquisition phase of the win-shift task. The results showed that the memory of vehicle treated rats was lost after more than thirty (>30) seconds in the test, while both the estradiol benzoate and 7-bromo-2-(4-hydroxyphenyl)-1,3-benzoxazol-5-ol treated rats demonstrated improved working memory in the test. These data indicate that ERβ agonism and not ERα agonism mimic the cognitive enhancing properties of estradiol benzoate. Thus, both estradiol benzoate and ERβ agonists augment cognition.

### EXAMPLE 5

### Evaluation of ERB Agonists on Synaptic Plasticity

In Experiment 1, ovariectomy carried out either five (5) days or five (5) weeks before testing impaired the induction of long-term depression (LTD), but not long-term potentiation (LTP). In Experiment 2, chronic estrogen replacement (0.2 ml 10 pg injection of 17β-estradiol every 48 hours) over the course of five (5) weeks enhanced the magnitude of paired-pulse induced LTD in the CA1 region, but had no effect on the induction of LTP. The results demonstrate that acute and chronic estrogen deprivation disrupted dynamic synaptic plasticity processes in the hippocampal CA1 region and that this disruption was ameliorated by chronic estrogen replacement. The findings are discussed with reference to (1) the contribution of Ca²⁺ regulated synaptic signaling pathways in the CA1 region to estradiol modulation of LTP and LTD and (2) the potential functional significance of ovariectomy-induced changes in synaptic plasticity for learning and memory processes.

A restoration of plasticity in ovariectomized rats with an ERβ agonist on restoration of long-term depression would demonstrate that the compound is active on cellular models of memory and hippocampal hypofunctioning, and thus learning and memory. Restoration of impaired synaptic plasticity of ovariectomized female rats following estrogen treatment can be evaluated by the protocol used by Day and Good, January 2005, Neurobiol Learn Mem., 83(1): 13-21.

### EXAMPLE 6

### Evaluation of ERβ Agonists on Attention Function by the Five Choice Serial Reaction Time Test

The Continuous Performance Test (CPT) measures attention in humans. The CPT has been widely used in clinical research and has been demonstrated to be sensitive in detecting attention deficits across several disorders such as mild cognitive impairment, schizophrenia, Alzheimer's disease and Attention-Deficit Hyperactivity Disorder (ADHD). In ADHD, the CPT test has been used to assay attention processes such as vigilance and response control. ADHD children under such test conditions show over all lower performance as measured by increased impulsive and incorrect responding. The now well-established 5-choice serial reaction time (5CSRT) task is a useful pre-clinical tool to differentiate and characterize the effects of potential therapies on attentional function. The basic requirements of the 5CSRT test are similar to the CPT; the animal has to visually scan a set of 5 openings in one of which a light will flash for a brief period of time (e.g., 500 m/second). A nose-poke in the illuminated port is a correct response and is reinforced by the delivery of a food pellet to the magazine. An incorrect nose-poke is followed by a period of darkness. Generally rats receive up to 100 trials in a 30-minute period. Like its clinical counterpart, the CPT, several measures can be taken from the 5CSRT, including attention, executive functioning, impulsivity and hyperactivity. The performance of the rats can be delineated into different measures. For example, measures reflecting attention include: the number of correct trials, percent correct and missed trials. Premature responding is a measure of impulsivity while correct latency and magazine latency can indicate changes in activity and motivation. Manipulations of testing parameters in the 5CSRT can be used to alter levels of impulsivity and attention in order to allow for assessment of various pharmacological agents. Impulsivity can be dramatically increased, with a concomitant modest decrease in attention, by making the schedule of stimuli presentations unpredictable (i.e., varying the interval between trials in which the light stimulus is presented).

Prior to drug treatments, rats were trained to discriminate a brief visual stimulus presented randomly in one of the 5 spatial locations. At the beginning of each test session, the house light was illuminated and free delivery of a single food pellet to the magazine was made. Trial initiation was triggered when the rat opened the magazine to collect this pellet. After a fixed 5 seconds inter-trial interval (ITI), the light at the rear of one of the 5 openings was illuminated for 500 m/second. A nose-poke in this opening during illumination and for 5 seconds afterwards was reinforced by the delivery of a food pellet and a correct response was recorded. A response in a non-illuminated opening during the signal period (incorrect response) and failures to respond within the limited hold period (missed trial) were followed by a period of darkness. Premature responses, those nose-pokes into apertures prior to illumination, reset the ITI. Results: In this experiment the ERB agonist increased attention (30 mg/kg) after 3 days of treatment.

### EXAMPLE 7

### Evaluation of ERB Agonists on Working and Episodic Memory by the Watermaze Test

Use of this standard behavioral test for spatial reference memory following treatment with an ERβ agonist can evaluate working and episodic memory, and thus the effective activity of the compound on neurocognitive deficits.

### EXAMPLE 8

### Evaluation of ERβ Agonists on Novel Object Recognition

Novel Object Recognition is impaired in several disorders of memory including Alzheimer's disease, schizophrenia, mild cognitive impairment (MCI), stroke, amongst others. In rodents, norepinephrine is used extensively to examine the effects of drugs on this form of memory. An ERβ agonist, 7-bromo-2-(4-hydroxyphenyl)-1,3-benzoxazol-5-ol, was tested on this form of memory. In the habituation stage (Day 1), the rats, male Long Evans are habituated for 10 minutes each to an arena that contains 2 identical objects (YY). On trial 1 (Day 2), the arena is set up with a different set of identical objects (e.g., BB) and the animals are allowed to spend 5 minutes sniffing each objects. Thirty (30) minutes prior to T1, animals were injected with 7-bromo-2-(4-hydroxyphenyl)-1,3-benzoxazol-5-ol. Results: 7-bromo-2-(4-hydroxyphenyl)-1,3-benzoxazol-5-ol (0.5 mg/kg) demonstrated that on Trial 2 (48 hours after Day 2) the amount of time investigating the novel object versus the familiar object in Trial 2 is significantly improved.

### EXAMPLE 9

### Evaluation of ERβ Agonists on Mild Cognitive Impairment

cAMP-response-element-binding protein (CREB) is expressed in all cells in the brain and is a member of a family of proteins that function as transcription factors. CREB has been shown to be involved in processes such as the induction of long-term potentiation or depression of synaptic strength, the growth and formation of new synaptic connections, and protein synthesis-dependent processes involved in the retrieval and consolidation of memory. Aged animals show marked decreases in CREB activation and memory; and this decline in cognition in aged rats is a useful model of mild cognitive impairment seen in humans. As such, an ERβ agonist, 7-bromo-2-(4-hydroxyphenyl)-1,3-benzoxazol-5-ol, was tested on this form of recognition memory. Twenty (20) aged rats received 7-bromo-2-(4-hydroxyphenyl)-1,3-benzoxazol-5-ol at 1 mg/kg or vehicle 30 minutes prior to testing in the Novel Object Recognition (NOR) method (same protocol as Example 8). The older aged rats (15 months) had significantly lower CREB and memory levels (as tested in the NOR method)compared to young 3 month-old control rats. Results:A single injection of the 7-bromo-2-(4-hydroxyphenyl)-1,3-benzoxazol-5-ol restored the CREB levels and increased memory in these aged rats compared to those of young 3 month old rats.

Those skilled in the art will recognize that various changes and/or modifications may be made to aspects or embodiments of this invention and that such changes and/or modifications may be made without departing from the spirit of this invention. Therefore it is intended that the appended claims cover all such equivalent variations as will fall within the spirit and scope of this invention. Each reference cited in the present application, including literature references, books, patents and patent applications, is incorporated herein by reference in its entirety.

What is claimed is:

1. A method for treating Parkinson's disease comprising the steps of:
a) identifying a patient having said disease; and
b) administering to said patient a therapeutically effective amount of an ERβ selective ligand, wherein said ERβ selective ligand is substantially free of ERβ antagonist activity.

2. The method of claim 1 wherein the ERβ selective ligand has the Formula I: 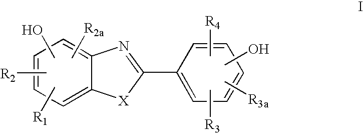
wherein:
R₁ is hydrogen, hydroxyl, halogen, alkyl of 1-6 carbon atoms, trifluoroalkyl of 1-6 carbon atoms, cycloalkyl of 3-8 carbon atoms, alkoxy of 1-6 carbon atoms, trifluoroalkoxy of 1-6 carbon atoms, thioalkyl of 1-6 carbon atoms, sulfoxoalkyl of 1-6 carbon atoms, sulfonoalkyl of 1-6 carbon atoms, aryl of 6-10 carbon atoms, a 5 or 6-membered heterocyclic ring having 1 to 4 heteroatoms selected from O, N or S, —NO₂, —NR₅R₆, —N(R₅)COR₆, —CN, —CHFCN, —CF₂CN, alkynyl of 2-7 carbon atoms, or alkenyl of 2-7 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₂ and R₂ₐ are each, independently, hydrogen, hydroxyl, halogen, alkyl of 1-6 carbon atoms, alkoxy of 1-4 carbon atoms, alkenyl of 2-7 carbon atoms, alkynyl of 2-7 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₃, R₃ₐ, and R₄ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₅ or R₆ are each, independently, hydrogen, alkyl of 1-6 carbon atoms, aryl of 6-10 carbon atoms;
X is O, S, or N R₇;
R₇ is hydrogen, alkyl of 1-6 carbon atoms, aryl of 6-10 carbon atoms, —COR₅, —CO₂R₅ or —SO₂R₅;
or a pharmaceutically acceptable salt or prodrug thereof.

3. The method of claim 2 wherein the ERβ selective ligand has the Formula II: 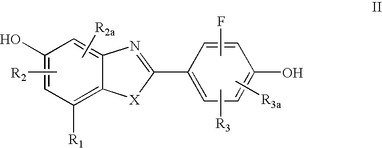
wherein:
R₁ is alkenyl of 2-7 carbon atoms; wherein the alkenyl moiety is optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₂ and R₂ₐ are each, independently, hydrogen, hydroxyl, halogen, alkyl of 1-6 carbon atoms, alkoxy of 1-4 carbon atoms, alkenyl of 2-7 carbon atoms, alkynyl of 2-7 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₃, and R₃ₐ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₅ or R₆ are each, independently, hydrogen, alkyl of 1-6 carbon atoms, aryl of 6-10 carbon atoms;
X is O, S, or N R₇;
R₇ is hydrogen, alkyl of 1-6 carbon atoms, aryl of 6-10 carbon atoms, —COR₅, —CO₂R₅ or —SO₂R₅;
or a pharmaceutically acceptable salt or prodrug thereof.

4. The method of claim 3 wherein X is O.

5. The method of claim 4 wherein R₁ is alkenyl of 2-3 carbon atoms, which is optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆.

6. The method of claim 3 wherein the ERβ selective ligand is 2-(3-fluoro-4-hydroxyphenyl)-7-vinyl-1,3-benzoxazol-5-ol or a pharmaceutically acceptable salt or prodrug thereof.

7. The method of claim 1 wherein the ERβ selective ligand has the Formula III: 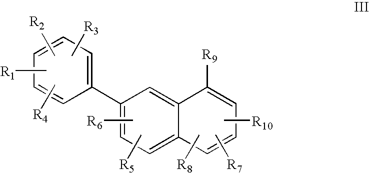
wherein:
R₁, R₂, R₃, and R₄ are each, independently, selected from hydrogen, hydroxyl, alkyl of 1-6 carbon atoms, alkoxy of 1-6 carbon atoms, or halogen;
R₅, R₆, R₇, R₈, R₉, and R₁₀ are each, independently, hydrogen, alkyl of 1-6 carbon atoms, alkenyl of 2-7 carbon atoms, alkynyl of 2-7 carbon atoms, halogen, alkoxy of 1-6 carbon atoms, —CN, —CHO, phenyl, or a 5 or 6-membered heterocyclic ring having 1 to 4 heteroatoms selected from O, N or S; wherein the alkyl or alkenyl moieties of R₅, R₆, R₇, R₈, R₉, or R₁₀ may be optionally substituted with hydroxyl, —CN, halogen, trifluoroalkyl, trifluoroalkoxy, —NO₂, or phenyl; wherein the phenyl moiety of R₅, R₆, R₇, R₈, R₉, or R₁₀ may be optionally mono-, di-, or tri-substituted with alkyl of 1-6 carbon atoms, alkenyl of 2-7 carbon atoms, halogen, hydroxyl, alkoxy of 1-6 carbon atoms, —CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;
wherein at least one of R₁, R₂, R₃, R₄, R₇, R₈, R₉, or R₁₀ is hydroxyl, or a pharmaceutically acceptable salt or prodrug thereof.

8. The method of claim 7 wherein the ERβ selective ligand has the Formula IV: 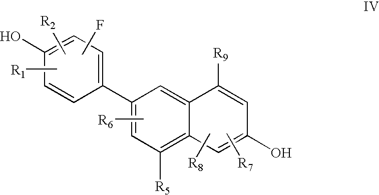
wherein:
R₁ and R₂ are each, independently, selected from hydrogen, hydroxyl, alkyl of 1-6 carbon atoms, alkenyl of 2-7 carbon atoms, and alkynyl of 2-7 carbon atoms, alkoxy of 1-6 carbon atoms, or halogen;
R₅, R₆, R₇, R₈, or R₉ are each, independently, hydrogen, alkyl of 1-6 carbon atoms, alkenyl of 2-7 carbon atoms, alkynyl of 2-7 carbon atoms, halogen, alkoxy of 1-6 carbon atoms, —CN, —CHO, trifluoromethyl, phenylalkyl of 7-12 carbon atoms, phenyl, or a 5 or 6-membered heterocyclic ring having 1 to 4 heteroatoms selected from O, N or S; wherein the alkyl or alkenyl moieties of R₅, R₆, R₇, R₈, or R₉ may be optionally substituted with hydroxyl, —CN, halogen, trifluoroalkyl, trifluoroalkoxy, —NO₂, or phenyl; wherein the phenyl moiety of R₅, R₆, R₇, R₈, or R₉ may be optionally mono-, di-, or tri-substituted with alkyl of 1-6 carbon atoms, alkenyl of 2-7 carbon atoms, halogen, hydroxyl, alkoxy of 1-6 carbon atoms, —CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;
wherein at least one of R₅ or R₉ is not hydrogen, or a pharmaceutically acceptable salt or prodrug thereof.

9. The method of claim 8 wherein the ERβ selective ligand has the Formula V: 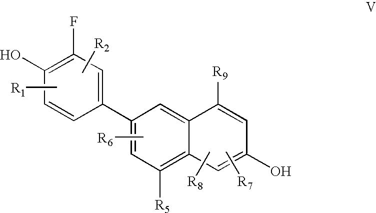
or a pharmaceutically acceptable salt or prodrug thereof.

10. The compound of claim 9 wherein the 5 or 6-membered heterocyclic ring having 1 to 4 heteroatoms selected from O, N or S is furan, thiophene or pyridine, or a pharmaceutically acceptable salt or prodrug thereof.

11. The compound of claim 10 wherein R₅, R₆, R₇, R₈, and R₉ are each, independently, hydrogen, halogen, —CN, alkynyl of 2-7 carbon atoms, or a pharmaceutically acceptable salt or prodrug thereof.

12. The compound of claim 11 wherein R₆, R₇, and R₈ are hydrogen or a pharmaceutically acceptable salt or prodrug thereof.

13. The compound of claim 8, wherein the compound of Formula IV is
a) 7-(4-hydroxyphenyl)-2-naphthol;
b) 7-(3-hydroxyphenyl)-2-naphthol;
c) 6-(4-hydroxyphenyl)-1-naphthol;
d) 6-phenyl-2-naphthol;
e) 6-(3-hydroxyphenyl)-2-naphthol;
f) 6-(3-chlorophenyl)-2-naphthol;
g) 2-fluoro-4-(2-naphthyl)phenol;
h) 6-(3-fluoro-4-hydroxyphenyl)-2-naphthol;
i) 6-(3-chloro-4-hydroxyphenol)-2-naphthol;
j) 1-chloro-6-phenyl-2-naphthol;
k) 1-bromo-6-(4-hydroxyphenyl)-2-naphthol;
l) 1 -chloro-6-(4-hydroxyphenyl)-2-naphthol;
m) 1-fluoro-6-(4-hydroxyphenyl)-2-naphthol;
n) 2-hydroxy-6-(4-hydroxyphenyl)-1-naphthonitrile;
o) 6-(4-hydroxyphenyl)-1-phenyl-2-naphthol;
p) 6-(4-hydroxyphenyl)-1-methyl-2-naphthol;
q) 1 -chloro-6-(3-fluoro-4-hydroxyphenyl)-2-naphthol;
r) 1 -chloro-6-(3-chloro-4-hydroxyphenyl)-2-naphthol;
s) 6-(4-hydroxyphenyl)-1-nitro-2-naphthol;
t) 1-chloro-6-(4-hydroxy-2-methylphenyl)-2-naphthol;
u) 6-(4-hydroxy-2-methylphenyl)-2-naphthol;
v) 6-(4-hydroxy-2-methoxyphenyl)-2-naphthol;
w) 6-(2-chloro-4-hydroxyphenyl)-2-naphthol;
x) 1-chloro-6-(2-chloro-4-hydroxyphenyl)-2-naphthol;
y) 6-(2-fluoro-4-hydroxyphenyl)-2-naphthol;
z) 6-(2,5-difluoro-4-hydroxyphenyl)-2-naphthol;
aa) 6-(2,6-difluoro-4-hydroxyphenyl)-2-naphthol;
bb) 1-chloro-6-(2-fluoro-4-hydroxyphenyl)-2-naphthol;
cc) 1-chloro-6-(2,5-difluoro-4-hydroxyphenyl)-2-naphthol;
dd) 1-chloro-6-(2,6-difluoro-4-hydroxyphenyl)-2-naphthol;
ee) 8-fluoro-6-(4-hydroxyphenyl)-2-naphthol;
ff) 1-chloro-8-fluoro-6-(4-hydroxyphenyl)-2-naphthol;
gg) 8-chloro-6-(4-hydroxyphenyl)-2-naphthol;
hh) 1,5-dichloro-8-fluoro-6-(4-hydroxyphenyl)-2-naphthol;
ii) 2-chloro-4-(2-naphthyl)phenol;
ij) 3-bromo-8-chloro-6-(4-hydroxyphenyl)-2-naphthol;
kk) 1,8-dichloro-6-(4-hydroxyphenyl)-2-naphthol;
II) 3-bromo-1,8-dichloro-6-(4-hydroxyphenyl)-2-naphthol;
mm) 7-hydroxy-3-(4-hydroxyphenyl)-1-naphthonitrile;
nn) 8-chloro-3-(4-hydroxyphenyl)-7-hydroxy-1-naphthonitrile;
oo) 8-chloro-3-(3-fluoro-4-hydroxyphenyl)-7-hydroxy-1-naphthonitrile;
pp) 6-(3,5-difluoro-4-hydroxyphenyl)-2-naphthol;
qq) 1 -chloro-6-(3,5-difluoro-4-hydroxyphenyl)-2-naphthol;
rr) 8-bromo-7-hydroxy-3-(4-hydroxyphenyl)-1-naphthonitrile;
ss) 8-fluoro-6-(3-fluoro-4-hydroxyphenyl)-2-naphthol;
tt) 1-chloro-8-fluoro-6-(3-fluoro-4-hydroxyphenyl)-2-naphthol;
uu) 3-(3-fluoro-4-hydroxyphenyl)-7-hydroxy-1-naphthonitrile;
vv) 3-(3,5-difluoro-4-hydroxyphenyl)-7-hydroxy-1-naphthonitrile;
or a pharmaceutically acceptable salt or prodrug thereof.

14. The method of claim 1 wherein the ERβ selective ligand has the Formula VI: 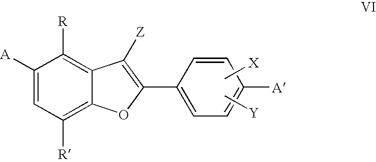
wherein:
A is alkyl of 1-6 carbon atoms, halogen, trifluoroalkyl of 1-6 carbon atoms, hydroxyalkyl of 1-6 carbon atoms, —CO₂H, —NH₂, or —OP;
A′ is —OP, —CO₂P, halogen, or hydroxyalkyl;
P is hydrogen, alkyl of 1-6 carbon atoms, or phenyl;
Z is hydrogen, alkyl of 1-6 carbon atoms, halogen, —NO₂, —CN, trifluoroalkyl of 1-6 carbon atoms, —COP, —CO₂P, or —C(P)═N—OP;
R and R′ are each, independently, hydrogen, alkyl of 1-6 carbon atoms, alkenyl of 2-7 carbon atoms, halogen, —OP, —SP, —SOP, —SO₂P, —SCN, trifluoroalkyl of 1-6 carbon atoms, —CF₂CF₃, trifluoroalkoxy of 1-6 carbon atoms, —NO₂, —NH₂, —NHOP, hydroxyalkyl of 1-6 carbon atoms, alkoxyalkyl of 1-6 carbon atoms per alkyl group, -alkyl-SP, -alkyl-SOP, -alkyl-SO₂P, —CN, -alkyl-CN, -alkenyl-CN, -alkylSCN, —CHFCN, —CF₂CN, -alkenyl-NO₂, haloalkyl of 1-6 carbon atoms, dihaloalkenyl of 2-7 carbon atoms, —COP, —COCF₃, —CO₂P, —CONR₁R₂, -alkyl-CONR, R₂, -alkenyl-CONR₁R₂, -alkyl-COP, -alkenyl-COP, -alkenyl-CO₂P, -alkenyl-CO₂P, oxadiazolyl, furyl, thienyl, pyrrolyl, imidazolyl, triazolyl, or tetrazolyl;
X and Y are each, independently, hydrogen, alkyl of 1-6 carbon atoms, halogen, —NO₂, —CN, trifluoroalkyl of 1-6 carbon atoms, —OP, hydroxyalkyl of 1-6 carbon atoms, —CO₂H, or phenyl which is optionally mono- or di-substituted with hydroxyl, benzyloxy, alkoxy of 1-6 carbon atoms, or —OCH₂CH₂NR₁R₂;
R₁ and R₂ are each, independently, hydrogen, alkyl of 1-6 carbon atoms, or alkoxy of 1-6 carbon atoms; or R₁ and R₂ are concatenated together as —(CH₂)ₚ—;
p=2-6;
or a pharmaceutically acceptable salt or prodrug thereof.

15. The method of claim 1 wherein the ER,8 selective ligand has of Formula VII: 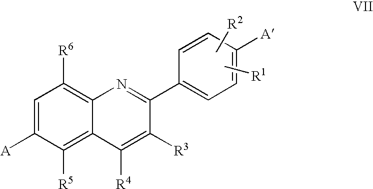
wherein:
A and A′ are each, independently, OH or OP;
P is alkyl, alkenyl, benzyl, acyl, aroyl, alkoxycarbonyl, sulfonyl or phosphoryl;
R¹ and R² are each, independently, H, halogen, C₁-C₆ alkyl, C₂-C₇ alkenyl, or C₁-C₆ alkoxy;
R³ is H, halogen, or C₁-C₆ alkyl;
R⁴ is H, halogen, C₁-C₆ alkyl, C₂-C₇ alkenyl, C₂-C₇ alkynyl, C₃-C₇ cycloalkyl, C₁-C₆ alkoxy, —CN, —CHO, acyl, or heteroaryl;
R⁵ and R⁶ are each, independently, H, halogen, C₁-C₆ alkyl, C₂-C₇ alkenyl, C₂-C₇ alkynyl, C₃-C₇ cycloalkyl, C₁-C₆ alkoxy, —CN, —CHO, acyl, phenyl, aryl or heteroaryl, provided that at least one of R⁴, R⁵ and R⁶ is halogen, C₁-C₆ alkyl, C₂-C₇ alkenyl, C₂-C₇ alkynyl, C₃-C₇ cycloalkyl, C₁-C₆ alkoxy, —CN, —CHO, acyl, phenyl, aryl or heteroaryl;
wherein the alkyl or alkenyl moieties of R⁴, R⁵ or R⁶ may be optionally substituted with halogen, OH, —CN, trifluoroalkyl, trifluoroalkoxy, —NO₂, or phenyl;
wherein the alkynyl moiety of R⁴, R⁵ or R⁶ may be optionally substituted with halogen, —CN, —CHO, acyl, trifluoroalkyl, trialkylsilyl, or optionally substituted phenyl;
wherein the phenyl moiety of R⁵ or R⁶ may be optionally mono-, di-, or tri-substituted with halogen, C₁-C₆ alkyl, C₂-C₇ alkenyl, OH, C₁-C₆ alkoxy, —CN, —CHO, —NO₂, amino, C₁-C₆ alkylamino, di-(C₁-C₆)alkylamino, thiol, or C₁-C₆ alkylthio;
provided that when each of R⁴, R⁵ and R⁶ are H, C₁-C₆ alkyl, C₂-C₇ alkenyl, or C₁-C₆ alkoxy, then at least one of R¹ and R² is halogen, C₁-C₆ alkyl, C₂-C₇ alkenyl, or C₁-C₆ alkoxy;
provided that at least one of R⁴ and R⁶ is other than H;
or a N-oxide thereof;
or Formula VIII: 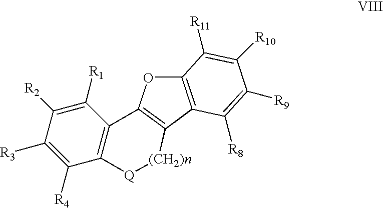
wherein:
Q has the structure i, ii or iii: 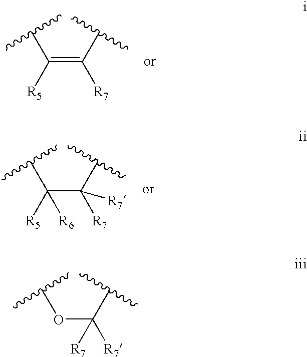
R₁, R₄, R₅, R₆ R₇, R_{7′}, R₈ and R₉, are each independently selected from the group consisting of hydrogen, C₁-C₆ alkyl, —OR₂₀, halogen, —CF₃, —CF₂CF₃, —CH₂CF₃, —SR₂₀, NR₂₀R₂₁, —CN, —CH₂CN, —CH₂CH₂CN, —CH═CHCN, —NO₂, —CH₂NO₂, —CH₂CH₂NO₂, —CH═CHNO₂ and —COR₂₀;
n=0 or 1;
each R₂₀ and R₂₁ is independently selected from the group consisting of hydrogen, C₁-C₆ alkyl, —CF₃, benzyl, —CO₂(C₁-C₆ alkyl) and —CO(C₁-C₆ alkyl);
provided that:
a) one of R₂ or R₃ must be —OR₂₀;
b) one of R₉ or R₁₀ must be —OR₂₀;
c) when R₂ is —OR₂₀, then R₁ and R₃ are selected independently from the group consisting of hydrogen, halogen, C₁-C6 alkyl, —CF₃, —CF₂CF₃, —CH₂CF₃, —SR₂₀, —CN, —CH₂CN, —CH₂CH₂CN, —CH═CHCN, —NO₂, —CH₂NO₂, —CH₂CH₂NO₂, —CH═CHNO₂ and —COR₂₀;
d) when R₃ is —OR₂₀, then R₂ and R₄ are selected independently from the group consisting of hydrogen, C₁-C₆ alkyl, halogen, —CF₃, —CF₂CF₃, —CH₂CF₃, —SR₂₀, —CN, —CH₂CN, —CH₂CH₂CN, —CH═CHCN, —NO₂, —CH₂NO₂, —CH₂CH₂NO₂, —CH═CHNO₂ and —COR₂₀;
e) when R₉ is —OR₂₀, then R₈ and R₁₀ are selected independently from the group consisting of hydrogen, C₁-C₆ alkyl, halogen, —CF₃, —CF₂CF₃, —CH₂CF₃, —SR₂₀, —CN, —CH₂CN, —CH₂CH₂CN, —CH═CHCN, —NO₂, —CH₂NO₂, —CH₂CH₂NO₂, —CH═CHNO₂ and —COR₂₀;
f) when R₁₀ is —OR₂₀, then R₉ and R₁₁ are selected independently from the group consisting of hydrogen, C₁-C₆ alkyl, halogen, —CF₃, —CF₂CF₃, —CH₂CF₃, —SR₂₀, —CN, —CH₂CN, —CH₂CH₂CN, —CH═CHCN, —NO₂, —CH₂NO₂, —CH₂CH₂NO₂, —CH═CHNO₂ and —COR₂₀; and
g) when Q has the structure iii, and R₇, R₇, R₈, R₉, R₁₁, are each H, and n=0, then R₁₀ is not OR₂₀,
or a pharmaceutically acceptable salt or prodrug thereof;
or Formula IX: 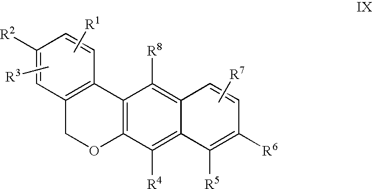
wherein:
R₁, R₂, R₃, R₅, R₆, R₇, and R₈ are each, independently, selected from hydrogen, hydroxyl, C₁-C₆ alkyl, C₁-C₆ alkoxy, or halogen;
R₄ is hydrogen, C₁-C₆ alkyl, halogen, C₁-C₆ alkoxy, —CN, C₂-C₈ alkenyl, —CHO, aryl, furyl, thienyl, pyrimidinyl, or pyridinyl;
provided that at least one of R.- R₈ is other than H;
or a pharmaceutically acceptable salt or prodrug thereof;
or of Formula X: 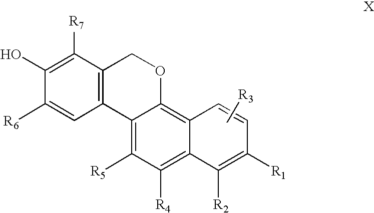
wherein:
R₁ and R₂ are each, independently, selected from hydrogen, hydroxyl, alkyl of 1-6 carbon atoms, alkenyl of 2-6 carbon atoms, alkynyl of 2-7 carbon atoms, alkoxy of 1-6 carbon atoms, or halogen; wherein the alkyl or alkenyl moieties of R₁ or R₂ may be optionally substituted with hydroxyl, —CN, halogen, trifluoroalkyl, trifluoroalkoxy, —NO₂, or phenyl; and provided that at least one of R₁ or R₂ is hydroxyl;
R₃, R₄, R₅, R₆, and R₇ are each, independently, hydrogen, alkyl of 1-6 carbon atoms, halogen, alkoxy of 1-6 carbon atoms, —CN, alkenyl of 2-7 carbon atoms, alkynyl of 2-7 carbon atoms, —CHO, phenyl, or a 5 or 6-membered heterocyclic ring having 1 to 4 heteroatoms selected from O, N or S; wherein the alkyl or alkenyl moieties of R₄, R₅, R₆, or R₇ may be optionally substituted with hydroxyl, —CN, halogen, trifluoroalkyl, trifluoroalkoxy, —NO₂, or phenyl; wherein the phenyl moiety of R₄ or R₅ may be optionally mono-, di-, or tri-substituted with alkyl of 1-6 carbon atoms, alkenyl of 2-7 carbon atoms, halogen, hydroxyl, alkoxy of 1-6 carbon atoms, —CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;
or a pharmaceutically acceptable salt or prodrug thereof.

16. A method for ameliorating a symptom of Parkinson's disease comprising the steps of:
a) identifying a patient having said disease and having said symptom thereof; and
b) administering to said patient a therapeutically effective amount of an ERβ selective ligand, wherein said ERβ selective ligand is substantially free of ERβ antagonist activity.

17. The method of claim 16 wherein the ERβ selective ligand has the Formula I: 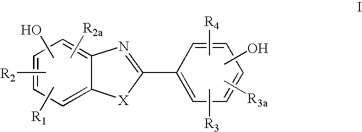
wherein:
R₁ is hydrogen, hydroxyl, halogen, alkyl of 1-6 carbon atoms, trifluoroalkyl of 1-6 carbon atoms, cycloalkyl of 3-8 carbon atoms, alkoxy of 1-6 carbon atoms, trifluoroalkoxy of 1-6 carbon atoms, thioalkyl of 1-6 carbon atoms, sulfoxoalkyl of 1-6 carbon atoms, sulfonoalkyl of 1-6 carbon atoms, aryl of 6-10 carbon atoms, a 5 or 6-membered heterocyclic ring having 1 to 4 heteroatoms selected from O, N or S, —NO₂, —NR₅R₆, —N(R₅)COR₆, —CN, —CHFCN, —CF₂CN, alkynyl of 2-7 carbon atoms, or alkenyl of 2-7 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₂ and R₂ₐ are each, independently, hydrogen, hydroxyl, halogen, alkyl of 1-6 carbon atoms, alkoxy of 1-4 carbon atoms, alkenyl of 2-7 carbon atoms, alkynyl of 2-7 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₃, R₃ₐ, and R₄ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₅ or R₆ are each, independently, hydrogen, alkyl of 1-6 carbon atoms, aryl of 6-10 carbon atoms;
X is O, S, or NR₇;
R₇ is hydrogen, alkylbof 1-6 carbon atoms, aryl of 6-10 carbon atoms, —COR₅, —CO₂R₅ or —SO₂R₅;
or a pharmaceutically acceptable salt or prodrug thereof.

18. The method of claim 17 wherein the ERβ selective ligand has the Formula II: 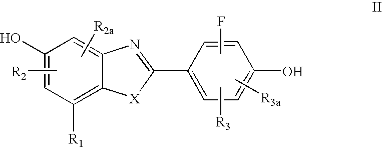
wherein:
R₁ is alkenyl of 2-7 carbon atoms; wherein the alkenyl moiety is optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₂ and R₂ₐ are each, independently, hydrogen, hydroxyl, halogen, alkyl of 1-6 carbon atoms, alkoxy of 1-4 carbon atoms, alkenyl of 2-7 carbon atoms, alkynyl of 2-7 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, -CN, halogen, trifluoroalkyl, trifluoroalkoxy, —COR₅, —CO₂R₅, —NO₂, CONR₅R₆, NR₅R₆ or N(R₅)COR₆;
R₃, and R₃ₐ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₅ or R₆ are each, independently, hydrogen, alkyl of 1-6 carbon atoms, aryl of 6-10 carbon atoms;
X is O, S, or N R₇;
R₇ is hydrogen, alkyl of 1-6 carbon atoms, aryl of 6-10 carbon atoms, —COR₅, —CO₂R₅ or —SO₂R₅;
or a pharmaceutically acceptable salt or prodrug thereof.

19. The method of claim 18 wherein X is O.

20. The method of claim 19 wherein R₁ is alkenyl of 2-3 carbon atoms, which is optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆.

21. The method of claim 18 wherein the ER,8 selective ligand is 2-(3-fluoro-4-hydroxyphenyl)-7-vinyl-1,3-benzoxazol-5-ol or a pharmaceutically acceptable salt or prodrug thereof.

22. The method of claim 16 wherein the ERβ selective ligand has the Formula III: 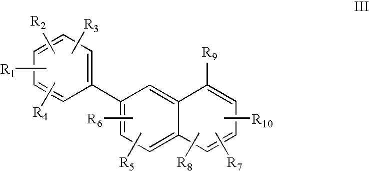
wherein:
R₁, R₂, R₃, and R₄ are each, independently, selected from hydrogen, hydroxyl, alkyl of 1-6 carbon atoms, alkoxy of 1-6 carbon atoms, or halogen;
R₅, R₆, R₇, R₈, R₉, and R₁₀ are each, independently, hydrogen, alkyl of 1-6 carbon atoms, alkenyl of 2-7 carbon atoms, alkynyl of 2-7 carbon atoms, halogen, alkoxy of 1-6 carbon atoms, —CN, —CHO, phenyl, or a 5 or 6-membered heterocyclic ring having 1 to 4 heteroatoms selected from 0, N or S; wherein the alkyl or alkenyl moieties of R₅, R₆, R₇, R₈, R₉, or R₁₀ may be optionally substituted with hydroxyl, —CN, halogen, trifluoroalkyl, trifluoroalkoxy, —NO₂, or phenyl; wherein the phenyl moiety of R₅, R₆, R₇, R₈, R₉, or R₁₀ may be optionally mono-, di-, or tri-substituted with alkyl of 1-6 carbon atoms, alkenyl of 2-7 carbon atoms, halogen, hydroxyl, alkoxy of 1-6 carbon atoms, —CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;
wherein at least one of R₁, R₂, R₃, R₄, R₇, R₈, R₉, or R₁₀ is hydroxyl, or a pharmaceutically acceptable salt or prodrug thereof.

23. The method of claim 22 wherein the ERβ selective ligand has the Formula IV: 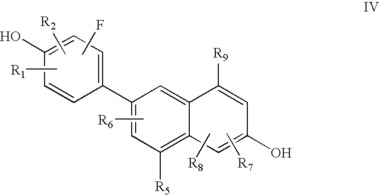
wherein:
R₁ and R₂ are each, independently, selected from hydrogen, hydroxyl, alkyl of 1-6 carbon atoms, alkenyl of 2-7 carbon atoms, and alkynyl of 2-7 carbon atoms, alkoxy of 1-6 carbon atoms, or halogen;
R₅, R₆, R₇, R₈, or R₉ are each, independently, hydrogen, alkyl of 1-6 carbon atoms, alkenyl of 2-7 carbon atoms, alkynyl of 2-7 carbon atoms, halogen, alkoxy of 1-6 carbon atoms, —CN, —CHO, trifluoromethyl, phenylalkyl of 7-12 carbon atoms, phenyl, or a 5 or 6-membered heterocyclic ring having 1 to 4 heteroatoms selected from O, N or S; wherein the alkyl or alkenyl moieties of R₅, R₆, R₇, R₈, or R₉ may be optionally substituted with hydroxyl, —CN, halogen, trifluoroalkyl, trifluoroalkoxy, —NO₂, or phenyl; wherein the phenyl moiety of R₅, R₆, R₇, R₈, or R₉ may be optionally mono-, di-, or tri-substituted with alkyl of 1-6 carbon atoms, alkenyl of 2-7 carbon atoms, halogen, hydroxyl, alkoxy of 1-6 carbon atoms, —CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;
wherein at least one of R₅ or R₉ is not hydrogen, or a pharmaceutically acceptable salt or prodrug thereof.

24. The method of claim 23 wherein the ERβ selective ligand has the Formula V: 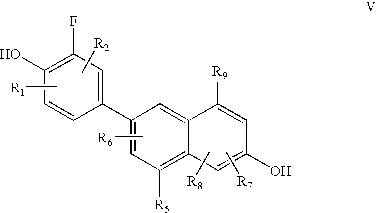
or a pharmaceutically acceptable salt or prodrug thereof.

25. The method of claim 24 wherein the 5 or 6-membered heterocyclic ring having 1 to 4 heteroatoms selected from O, N or S is furan, thiophene or pyridine, or a pharmaceutically acceptable salt or prodrug thereof.

26. The method of claim 25 wherein R₅, R₆, R₇, R₈, and R₉ are each, independently, hydrogen, halogen, —CN, alkynyl of 2-7 carbon atoms, alkoxy of 1-6 carbon atoms, —CHO, trifluoromethyl or phenylalkyl of 7-12 carbon atoms, or a pharmaceutically acceptable salt or prodrug thereof.

27. The method of claim 26 wherein R₆, R₇, and R₈ are hydrogen or a pharmaceutically acceptable salt or prodrug thereof.

28. The method of claim 23, wherein the compound of Formula IV is:
a) 7-(4-hydroxyphenyl)-2-naphthol;
b) 7-(3-hydroxyphenyl)-2-naphthol;
c) 6-(4-hydroxyphenyl)-1-naphthol;
d) 6-phenyl-2-naphthol;
e) 6-(3-hydroxyphenyl)-2-naphthol;
f) 6-(3-chlorophenyl)-2-naphthol;
g) 2-fluoro-4-(2-naphthyl)phenol;
h) 6-(3-fluoro-4-hydroxyphenyl)-2-naphthol;
i) 6-(3-chloro-4-hydroxyphenol)-2-naphthol;
j) 1-chloro-6-phenyl-2-naphthol;
k) 1-bromo-6-(4-hydroxyphenyl)-2-naphthol;
l) 1-chloro-6-(4-hydroxyphenyl)-2-naphthol;
m) 1-fluoro-6-(4-hydroxyphenyl)-2-naphthol;
n) 2-hydroxy-6-(4-hydroxyphenyl)-1-naphthonitrile;
o) 6-(4-hydroxyphenyl)-1-phenyl-2-naphthol;
p) 6-(4-hydroxyphenyl)-1-methyl-2-naphthol;
q) 1-chloro-6-(3-fluoro-4-hydroxyphenyl)-2-naphthol;
r) 1-chloro-6-(3-chloro-4-hydroxyphenyl)-2-naphthol;
s) 6-(4-hydroxyphenyl)-1-nitro-2-naphthol;
t) 1-chloro-6-(4-hydroxy-2-methylphenyl)-2-naphthol;
u) 6-(4-hydroxy-2-methylphenyl)-2-naphthol;
v) 6-(4-hydroxy-2-methoxyphenyl)-2-naphthol;
w) 6-(2-chloro-4-hydroxyphenyl)-2-naphthol;
x) 1-chloro-6-(2-chloro-4-hydroxyphenyl)-2-naphthol;
y) 6-(2-fluoro-4-hydroxyphenyl)-2-naphthol;
z) 6-(2,5-difluoro-4-hydroxyphenyl)-2-naphthol;
aa) 6-(2,6-difluoro-4-hydroxyphenyl)-2-naphthol;
bb) 1-chloro-6-(2-fluoro-4-hydroxyphenyl)-2-naphthol;
cc) 1-chloro-6-(2,5-difluoro-4-hydroxyphenyl)-2-naphthol;
dd) 1-chloro-6-(2,6-difluoro-4-hydroxyphenyl)-2-naphthol;
ee) 8-fluoro-6-(4-hydroxyphenyl)-2-naphthol;
ff) 1-chloro-8-fluoro-6-(4-hydroxyphenyl)-2-naphthol;
gg) 8-chloro-6-(4-hydroxyphenyl)-2-naphthol;
hh) 1,5-dichloro-8-fluoro-6-(4-hydroxyphenyl)-2-naphthol;
ii) 2-chloro-4-(2-naphthyl)phenol;
jj) 3-bromo-8-chloro-6-(4-hydroxyphenyl)-2-naphthol;
kk) 1,8-dichloro-6-(4-hydroxyphenyl)-2-naphthol;
ll) 3-bromo-1,8-dichloro-6-(4-hydroxyphenyl)-2-naphthol;
mm) 7-hydroxy-3-(4-hydroxyphenyl)-1-naphthonitrile;
nn) 8-chloro-3-(4-hydroxyphenyl)-7-hydroxy-1-naphthonitrile;
oo) 8-chloro-3-(3-fluoro-4-hydroxyphenyl)-7-hydroxy-1-naphthonitrile;
pp) 6-(3,5-difluoro-4-hydroxyphenyl)-2-naphthol;
qq) 1-chloro-6-(3,5-difluoro-4-hydroxyphenyl)-2-naphthol;
rr) 8-bromo-7-hydroxy-3-(4-hydroxyphenyl)-1-naphthonitrile;
ss) 8-fluoro-6-(3-fluoro-4-hydroxyphenyl)-2-naphthol;
tt) 1-chloro-8-fluoro-6-(3-fluoro-4-hydroxyphenyl)-2-naphthol;
uu) 3-(3-fluoro-4-hydroxyphenyl)-7-hydroxy-1-naphthonitrile; or
vv) 3-(3,5-difluoro-4-hydroxyphenyl)-7-hydroxy-1-naphthonitrile;
or a pharmaceutically acceptable salt or prodrug thereof.

29. The method of claim 16 wherein the ERβ selective ligand has the Formula VI: 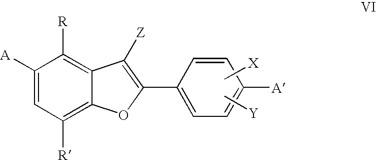
wherein:
A is alkyl of 1-6 carbon atoms, halogen, trifluoroalkyl of 1-6 carbon atoms, hydroxyalkyl of 1-6 carbon atoms, —CO₂H, —NH₂, or —OP;
A′ is —OP, —CO₂P, halogen, or hydroxyalkyl;
P is hydrogen, alkyl of 1-6 carbon atoms, or phenyl;
Z is hydrogen, alkyl of 1-6 carbon atoms, halogen, —NO₂, —CN, trifluoroalkyl of 1-6 carbon atoms, —COP, —CO₂P, or —C(P)═N—OP;
R and R′ are each, independently, hydrogen, alkyl of 1-6 carbon atoms, alkenyl of 2-7 carbon atoms, halogen, —OP, —SP, —SOP, —SO₂P, —SCN, trifluoroalkyl of 1-6 carbon atoms, —CF₂CF₃, trifluoroalkoxy of 1-6 carbon atoms, —NO₂, —NH₂, —NHOP, hydroxyalkyl of 1-6 carbon atoms, alkoxyalkyl of 1-6 carbon atoms per alkyl group, -alkyl-SP, -alkyl-SOP, -alkyl-SO₂P, —CN, -alkyl-CN, -alkenyl-CN, -alkylSCN, —CHFCN, —CF₂CN, -alkenyl-NO₂, haloalkyl of 1-6 carbon atoms, dihaloalkenyl of 2-7 carbon atoms, —COP, —COCF₃, —CO₂P, —CONR₁R₂, -alkyl-CONR, R₂, -alkenyl-CONR, R₂, -alkyl-COP, -alkenyl-COP, -alkenyl-CO₂P, -alkenyl-CO₂P, oxadiazolyl, furyl, thienyl, pyrrolyl, imidazolyl, triazolyl, or tetrazolyl;
X and Y are each, independently, hydrogen, alkyl of 1-6 carbon atoms, halogen, —NO₂, —CN, trifluoroalkyl of 1-6 carbon atoms, —OP, hydroxyalkyl of 1-6 carbon atoms, —CO₂H, or phenyl which is optionally mono- or di-substituted with hydroxyl, benzyloxy, alkoxy of 1-6 carbon atoms, or —OCH₂CH₂NR₁R₂;
R₁ and R₂ are each, independently, hydrogen, alkyl of 1-6 carbon atoms, or alkoxy of 1-6 carbon atoms; or R₁ and R₂ are concatenated together as —(CH₂)ₚ—;
p=2-6;
or a pharmaceutically acceptable salt or prodrug thereof.

30. The method of claim 16 wherein the ER,6 selective ligand has the Formula VII: 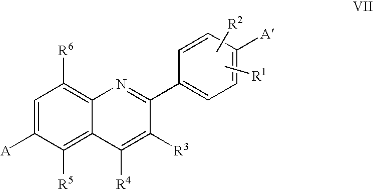
wherein:
A and A′ are each, independently, OH or OP;
P is alkyl, alkenyl, benzyl, acyl, aroyl, alkoxycarbonyl, sulfonyl or phosphoryl;
R¹ and R² are each, independently, H, halogen, C₁-C6 alkyl, C₂-C₇ alkenyl, or C₁-C₆ alkoxy;
R³ is H, halogen, or C₁-C₆ alkyl;
R⁴ is H, halogen, C₁-C₆ alkyl, C₂-C₇ alkenyl, C₂-C₇ alkynyl, C₃-C₇ cycloalkyl, C₁-C₆ alkoxy, —CN, —CHO, acyl, or heteroaryl;
R⁵ and R⁶ are each, independently, H, halogen, C₁-C₆ alkyl, C₂-C₇ alkenyl, C₂-C₇ alkynyl, C₃-C₇ cycloalkyl, C₁-C₆ alkoxy, —CN, —CHO, acyl, phenyl, aryl or heteroaryl, provided that at least one of R⁴, R⁵ and R⁶ is halogen, C₁-C₆ alkyl, C₂-C₇ alkenyl, C₂-C₇ alkynyl, C₃-C₇ cycloalkyl, C₁-C₆ alkoxy, —CN, —CHO, acyl, phenyl, aryl or heteroaryl;
wherein the alkyl or alkenyl moieties of R⁴, R⁵ or R⁶ may be optionally substituted with halogen, OH, —CN, trifluoroalkyl, trifluoroalkoxy, —NO₂, or phenyl;
wherein the alkynyl moiety of R⁴, R⁵ or R⁶ may be optionally substituted with halogen, —CN, —CHO, acyl, trifluoroalkyl, trialkylsilyl, or optionally substituted phenyl;
wherein the phenyl moiety of R⁵ or R⁶ may be optionally mono-, di-, or tri-substituted with halogen, C₁-C₆ alkyl, C₂-C₇ alkenyl, OH, C₁-C₆ alkoxy, —CN, —CHO, —NO₂, amino, C₁-C₆ alkylamino, di-(C₁-C₆)alkylamino, thiol, or C₁-C₆ alkylthio;
provided that when each of R⁴, R⁵ and R⁶ are H, C₁-C₆ alkyl, C₂-C₇ alkenyl, or C₁-C₆ alkoxy, then at least one of R¹ and R² is halogen, C₁-C₆ alkyl, C₂-C₇ alkenyl, or C₁-C₆ alkoxy;
provided that at least one of R⁴ and R⁶ is other than H;
or a N-oxide thereof;
or Formula VIII: 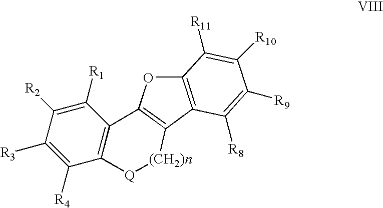
wherein:
Q has the structure i, ii or iii: 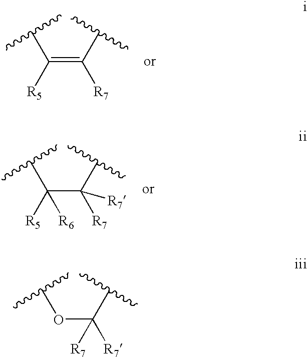
R₁, R₄, R₅, R₆ R₇, R_{7′}, R₈ and R₁₁ are each independently selected from the group consisting of hydrogen, C₁-C₆ alkyl, —OR₂₀, halogen, —CF₃, —CF₂CF₃, —CH₂CF₃, —SR₂₀, NR₂₀R₂₁, —CN, —CH₂CN, —CH₂CH₂CN, —CH═CHCN, —NO₂, —CH₂NO₂, —CH₂CH₂NO₂, —CH═CHNO₂ and —COR₂₀;
n=0 or 1;
each R₂₀ and R₂₁ is independently selected from the group consisting of hydrogen, C₁-C₆ alkyl, —CF₃, benzyl, —CO₂(C₁-C₆ alkyl) and —CO(C₁-C₆ alkyl); provided that:
a) one of R₂ or R₃ must be —OR₂₀;
b) one of R₉ or R₁₀ must be —OR₂₀;
c) when R₂ is —OR₂₀, then R₁ and R₃ are selected independently from the group consisting of hydrogen, halogen, C₁-C₆ alkyl, —CF₃, —CF₂CF₃, —CH₂CF₃, —SR₂₀, —CN, —CH₂CN, —CH₂CH₂CN, —CH═CHCN, —NO₂, —CH₂NO₂, —CH₂CH₂NO₂, —CH═CHNO₂ and —COR₂₀;
d) when R₃ is —OR₂₀, then R₂ and R₄ are selected independently from the group consisting of hydrogen, C₁-C₆ alkyl, halogen, —CF₃, —CF₂CF₃, —CH₂CF₃, —SR₂₀, —CN, —CH₂CN, —CH₂CH₂CN, —CH═CHCN, —NO₂, —CH₂NO₂, —CH₂CH₂NO₂, —CH═CHNO₂ and —COR₂₀;
e) when R₉ is —OR₂₀, then R₈ and R₁₀ are selected independently from the group consisting of hydrogen, C₁-C₆ alkyl, halogen, —CF₃, —CF₂CF₃, —CH₂CF₃, —SR₂₀, —CN, —CH₂CN, —CH₂CH₂CN, —CH═CHCN, —NO₂, —CH₂NO₂, —CH₂CH₂NO₂, —CH═CHNO₂ and —COR₂₀;
f) when R₁₀ is —OR₂₀, then R₉ and R₁₁ are selected independently from the group consisting of hydrogen, C₁-C₆ alkyl, halogen, —CF₃, —CF₂CF₃, —CH₂CF₃, —SR₂₀, —CN, —CH₂CN, —CH₂CH₂CN, —CH═CHCN, —NO₂, —CH₂NO₂, —CH₂CH₂NO₂, —CH═CHNO₂ and —COR₂₀; and
g) when Q has the structure iii, and R₇, R₇, R₈, R₉, R₁₁ are each H, and n=0, then R₁₀ is not OR₂₀,
or a pharmaceutically acceptable salt or prodrug thereof;
or Formula IX: 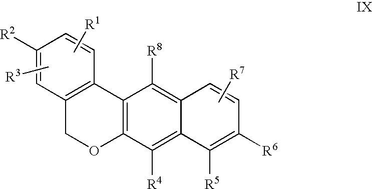
wherein:
R₁, R₂, R₃, R₅, R₆, R₇, and R₈ are each, independently, selected from hydrogen, hydroxyl, C₁-C₆ alkyl, C₁-C₆ alkoxy, or halogen;
R₄ is hydrogen, C₁-C₆alkyl, halogen, C₁-C₆alkoxy, —CN, C₂-C₈alkenyl, —CHO, aryl, furyl, thienyl, pyrimidinyl, or pyridinyl;
provided that at least one of R.- R₈ is other than H;
or a pharmaceutically acceptable salt or prodrug thereof;
or of Formula X: 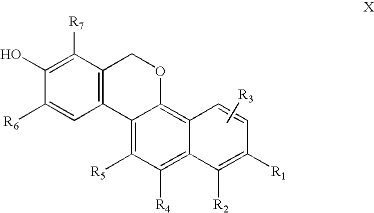
wherein:
R₁ and R₂ are each, independently, selected from hydrogen, hydroxyl, alkyl of 1-6 carbon atoms, alkenyl of 2-6 carbon atoms, alkynyl of 2-7 carbon atoms, alkoxy of 1-6 carbon atoms, or halogen; wherein the alkyl or alkenyl moieties of R₁ or R₂ may be optionally substituted with hydroxyl, —CN, halogen, trifluoroalkyl, trifluoroalkoxy, —NO₂, or phenyl; and provided that at least one of R₁ or R₂ is hydroxyl;
R₃, R₄, R₅, R₆, and R₇ are each, independently, hydrogen, alkyl of 1-6 carbon atoms, halogen, alkoxy of 1-6 carbon atoms, —CN, alkenyl of 2-7 carbon atoms, alkynyl of 2-7 carbon atoms, —CHO, phenyl, or a 5 or 6-membered heterocyclic ring having 1 to 4 heteroatoms selected from O, N or S; wherein the alkyl or alkenyl moieties of R₄, R₅, R₆, or R₇ may be optionally substituted with hydroxyl, —CN, halogen, trifluoroalkyl, trifluoroalkoxy, —NO₂, or phenyl; wherein the phenyl moiety of R₄ or R₅ may be optionally mono-, di-, or tri-substituted with alkyl of 1-6 carbon atoms, alkenyl of 2-7 carbon atoms, halogen, hydroxyl, alkoxy of 1-6 carbon atoms, —CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;
or a pharmaceutically acceptable salt or prodrug thereof.

31. The method of claim 16 wherein the symptom of Parkinson's disease is selected from the group consisting of poor balance, Parkinsonian gait, bradykinesia, rigidity, tremor, speech changes, loss of facial expression, micrographia, difficulty swallowing, drooling, pain, dementia or confusion, sleep disturbances, constipation, skin problems, depression, fear, anxiety, memory difficulties, and slowed thinking, sexual dysfunction, urinary problems, fatigue, aching, and loss of energy.

32. A method for ameliorating a symptom of a cognitive disease or disorder comprising the steps of:
a) identifying a patient having said cognitive disease or disorder and having said symptom thereof; and
b) administering to said patient a therapeutically effective amount of an ERβ selective ligand, wherein said ERβ selective ligand is substantially free of ERβ antagonist activity; and
wherein said disease or disorder is selected from multiple sclerosis, depression, schizophrenia, stroke, Alzheimer's disease or anxiety.

33. The method of claim 32 wherein the ERβ selective ligand has the Formula I: 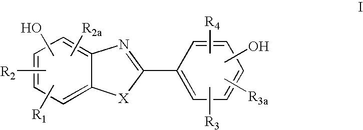
wherein:
R₁ is hydrogen, hydroxyl, halogen, alkyl of 1-6 carbon atoms, trifluoroalkyl of 1-6 carbon atoms, cycloalkyl of 3-8 carbon atoms, alkoxy of 1-6 carbon atoms, trifluoroalkoxy of 1-6 carbon atoms, thioalkyl of 1-6 carbon atoms, sulfoxoalkyl of 1-6 carbon atoms, sulfonoalkyl of 1-6 carbon atoms, aryl of 6-10 carbon atoms, a 5 or 6-membered heterocyclic ring having 1 to 4 heteroatoms selected from O, N or S, —NO₂, —NR₅R₆, —N(R₅)COR₆, —CN, —CHFCN, —CF₂CN, alkynyl of 2-7 carbon atoms, or alkenyl of 2-7 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₂ and R₂ₐ are each, independently, hydrogen, hydroxyl, halogen, alkyl of 1-6 carbon atoms, alkoxy of 1-4 carbon atoms, alkenyl of 2-7 carbon atoms, alkynyl of 2-7 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₃, R₃ₐ, and R₄ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₅ or R₆ are each, independently, hydrogen, alkyl of 1-6 carbon atoms, aryl of 6-10 carbon atoms;
X is O, S, or NR₇;
R₇ is hydrogen, alkyl of 1-6 carbon atoms, aryl of 6-10 carbon atoms, —COR₅, —CO₂R₅ or —SO₂R₅;
or a pharmaceutically acceptable salt or prodrug thereof.

34. The method of claim 33 wherein the ERβ selective ligand has the Formula II: 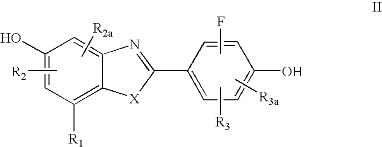
wherein:
R₁ is alkenyl of 2-7 carbon atoms; wherein the alkenyl moiety is optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₂ and R₂ₐ are each, independently, hydrogen, hydroxyl, halogen, alkyl of 1-6 carbon atoms, alkoxy of 1-4 carbon atoms, alkenyl of 2-7 carbon atoms, alkynyl of 2-7 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₃, and R₃ₐ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl, alkenyl, or alkynyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₅ or R₆ are each, independently, hydrogen, alkyl of 1-6 carbon atoms, aryl of 6-10 carbon atoms;
X is O, S, or NR₇;
R₇ is hydrogen, alkyl of 1-6 carbon atoms, aryl of 6-10 carbon atoms, —COR₅, —CO₂R₅ or —SO₂R₅;
or a pharmaceutically acceptable salt or prodrug thereof.

35. The method of claim 34 wherein X is O.

36. The method of claim 35 wherein R₁ is alkenyl of 2-3 carbon atoms, which is optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆.

37. The method of claim 34 wherein the ERβ selective ligand is 2-(3-fluoro-4-hydroxyphenyl)-7-vinyl-1,3-benzoxazol-5-ol or a pharmaceutically acceptable salt or prodrug thereof.

38. The method of claim 32 wherein the ERβ selective ligand has the Formula IlII 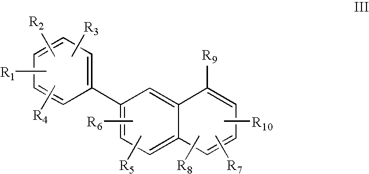
wherein:
R₁, R₂, R₃, and R₄ are each, independently, selected from hydrogen, hydroxyl, alkyl of 1-6 carbon atoms, alkoxy of 1-6 carbon atoms, or halogen;
R₅, R₆, R₇, R₈, R₉, and R₁₀ are each, independently, hydrogen, alkyl of 1-6 carbon atoms, alkenyl of 2-7 carbon atoms, alkynyl of 2-7 carbon atoms, halogen, alkoxy of 1-6 carbon atoms, —CN, —CHO, phenyl, or a 5 or 6-membered heterocyclic ring having 1 to 4 heteroatoms selected from O, N or S; wherein the alkyl or alkenyl moieties of R₅, R₆, R₇, R₈, R₉, or R₁₀ may be optionally substituted with hydroxyl, —CN, halogen, trifluoroalkyl, trifluoroalkoxy, —NO₂, or phenyl; wherein the phenyl moiety of R₅, R₆, R₇, R₈, R₉, or R₁₀ may be optionally mono-, di-, or tri-substituted with alkyl of 1-6 carbon atoms, alkenyl of 2-7 carbon atoms, halogen, hydroxyl, alkoxy of 1-6 carbon atoms, —CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;
wherein at least one of R₁, R₂, R₃, R₄, R₇, R₈, R₉, or R₁₀ is hydroxyl, or a pharmaceutically acceptable salt or prodrug thereof.

39. The method of claim 38 wherein the ERβ selective ligand has the Formula IV: 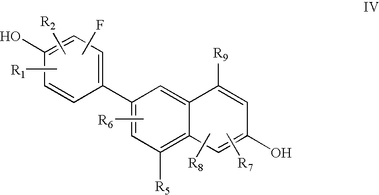
wherein:
R₁ and R₂ are each, independently, selected from hydrogen, hydroxyl, alkyl of 1-6 carbon atoms, alkenyl of 2-7 carbon atoms, and alkynyl of 2-7 carbon atoms, alkoxy of 1-6 carbon atoms, or halogen;
R₅, R₆, R₇, R₈, or R₉ are each, independently, hydrogen, alkyl of 1-6 carbon atoms, alkenyl of 2-7 carbon atoms, alkynyl of 2-7 carbon atoms, halogen, alkoxy of 1-6 carbon atoms, —CN, —CHO, trifluoromethyl, phenylalkyl of 7-12 carbon atoms, phenyl, or a 5 or 6-membered heterocyclic ring having 1 to 4 heteroatoms selected from O, N or S; wherein the alkyl or alkenyl moieties of R₅, R₆, R₇, R₈, or R₉ may be optionally substituted with hydroxyl, —CN, halogen, trifluoroalkyl, trifluoroalkoxy, —NO₂, or phenyl; wherein the phenyl moiety of R₅, R₆, R₇, R₈, or R₉ may be optionally mono-, di-, or tri-substituted with alkyl of 1-6 carbon atoms, alkenyl of 2-7 carbon atoms, halogen, hydroxyl, alkoxy of 1-6 carbon atoms, —CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;
wherein at least one of R₅ or R₉ is not hydrogen, or a pharmaceutically acceptable salt or prodrug thereof.

40. The method of claim 39 wherein the ERβ selective ligand has the Formula V: 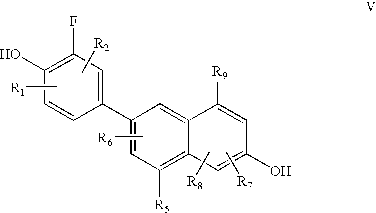
or a pharmaceutically acceptable salt or prodrug thereof.

41. The method of claim 40 wherein the 5 or 6-membered heterocyclic ring having 1 to 4 heteroatoms selected from O, N or S is furan, thiophene or pyridine, or a pharmaceutically acceptable salt or prodrug thereof.

42. The method of claim 41 wherein R₅, R₆, R₇, R₈, and Rg are each, independently, hydrogen, halogen, —CN, alkynyl of 2-7 carbon atoms, alkoxy of 1-6 carbon atoms, —CHO, trifluoromethyl or phenylalkyl of 7-12 carbon atoms, or a pharmaceutically acceptable salt or prodrug thereof.

43. The method of claim 42 wherein R₆, R₇, and R₈ are hydrogen or a pharmaceutically acceptable salt or prodrug thereof.

44. The method of claim 39 wherein the compound of Formula IV is:
ww) 7-(4-hydroxyphenyl)-2-naphthol;
xx) 7-(3-hydroxyphenyl)-2-naphthol;
yy) 6-(4-hydroxyphenyl)-1-naphthol;
zz) 6-phenyl-2-naphthol;
aaa) 6-(3-hydroxyphenyl)-2-naphthol;
bbb) 6-(3-chlorophenyl)-2-naphthol;
ccc) 2-fluoro-4-(2-naphthyl)phenol
ddd) 6-(3-fluoro-4-hydroxyphenyl)-2-naphthol;
eee) 6-(3-chloro-4-hydroxyphenol)-2-naphthol;
fff) 1-chloro-6-phenyl-2-naphthol;
ggg) 1-bromo-6-(4-hydroxyphenyl)-2-naphthol;
hhh) 1-chloro-6-(4-hydroxyphenyl)-2-naphthol;
iii) 1-fluoro-6-(4-hydroxyphenyl)-2-naphthol;
jjj) 2-hydroxy-6-(4-hydroxyphenyl)-1-naphthonitrile;
kkk) 6-(4-hydroxyphenyl)-1-phenyl-2-naphthol;
III) 6-(4-hydroxyphenyl)-1-methyl-2-naphthol;
mmm) 1-chloro-6-(3-fluoro-4-hydroxyphenyl)-2-naphthol;
nnn) 1-chloro-6-(3-chloro-4-hydroxyphenyl)-2-naphthol;
ooo) 6-(4-hydroxyphenyl)-1-nitro-2-naphthol;
ppp) 1-chloro-6-(4-hydroxy-2-methylphenyl)-2-naphthol;
qqq) 6-(4-hydroxy-2-methylphenyl)-2-naphthol;
rrr) 6-(4-hydroxy-2-methoxyphenyl)-2-naphthol;
sss) 6-(2-chloro-4-hydroxyphenyl)-2-naphthol;
ttt) 1-chloro-6-(2-chloro-4-hydroxyphenyl)-2-naphthol;
uuu) 6-(2-fluoro-4-hydroxyphenyl)-2-naphthol;
vvv) 6-(2,5-difluoro-4-hydroxyphenyl)-2-naphthol;
www) 6-(2,6-difluoro-4-hydroxyphenyl)-2-naphthol;
xxx) 1-chloro-6-(2-fluoro-4-hydroxyphenyl)-2-naphthol;
yyy) 1-chloro-6-(2,5-difluoro-4-hydroxyphenyl)-2-naphthol;
zzz) 1-chloro-6-(2,6-difluoro-4-hydroxyphenyl)-2-naphthol;
aaaa) 8-fluoro-6-(4-hydroxyphenyl)-2-naphthol;
bbbb) 1-chloro-8-fluoro-6-(4-hydroxyphenyl)-2-naphthol;
cccc) 8-chloro-6-(4-hydroxyphenyl)-2-naphthol;
dddd) 1,5-dichloro-8-fluoro-6-(4-hydroxyphenyl)-2-naphthol;
eeee) 2-chloro-4-(2-naphthyl)phenol;
ffff) 3-bromo-8-chloro-6-(4-hydroxyphenyl)-2-naphthol;
gggg) 1,8-dichloro-6-(4-hydroxyphenyl)-2-naphthol;
hhhh) 3-bromo-1,8-dichloro-6-(4-hydroxyphenyl)-2-naphthol;
iiii) 7-hydroxy-3-(4-hydroxyphenyl)-1-naphthonitrile;
jjj) 8-chloro-3-(4-hydroxyphenyl)-7-hydroxy-1-naphthonitrile;
kkkk) 8-chloro-3-(3-fluoro-4-hydroxyphenyl)-7-hydroxy-1-naphthonitrile;
lllll) 6-(3,5-difluoro-4-hydroxyphenyl)-2-naphthol;
mmmm) 1-chloro-6-(3,5-difluoro-4-hydroxyphenyl)-2-naphthol;
nnnn) 8-bromo-7-hydroxy-3-(4-hydroxyphenyl)- 1-naphthonitrile;
oooo) 8-fluoro-6-(3-fluoro-4-hydroxyphenyl)-2-naphthol;
pppp) 1-chloro-8-fluoro-6-(3-fluoro-4-hydroxyphenyl)-2-naphthol;
qqqq) 3-(3-fluoro-4-hydroxyphenyl)-7-hydroxy-1-naphthonitrile; or
rrrr) 3-(3,5-difluoro-4-hydroxyphenyl)-7-hydroxy-1-naphthonitrile;
or a pharmaceutically acceptable salt or prodrug thereof.

45. The method of claim 32 wherein the ERβ selective ligand has the Formula VI: 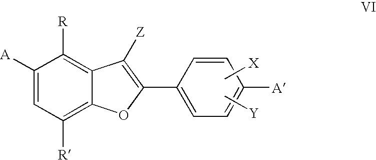
wherein:
A is alkyl of 1-6 carbon atoms, halogen, trifluoroalkyl of 1-6 carbon atoms, hydroxyalkyl of 1-6 carbon atoms, —CO₂H, —NH₂, or —OP;
A′ is —OP, —CO₂P, halogen, or hydroxyalkyl;
P is hydrogen, alkyl of 1-6 carbon atoms, or phenyl;
Z is hydrogen, alkyl of 1-6 carbon atoms, halogen, —NO₂, —CN, trifluoroalkyl of 1-6 carbon atoms, —COP, —CO₂P, or —C(P)═N—OP;
R and R′ are each, independently, hydrogen, alkyl of 1-6 carbon atoms, alkenyl of 2-7 carbon atoms, halogen, —OP, —SP, —SOP, —SO₂P, —SCN, trifluoroalkyl of 1-6 carbon atoms, —CF₂CF₃, trifluoroalkoxy of 1-6 carbon atoms, —NO₂, —NH₂, —NHOP, hydroxyalkyl of 1-6 carbon atoms, alkoxyalkyl of 1-6 carbon atoms per alkyl group, -alkyl-SP, -alkyl-SOP, -alkyl-SO₂P, —CN, -alkyl-CN, -alkenyl-CN, -alkylSCN, —CHFCN, —CF₂CN, -alkenyl-NO₂, haloalkyl of 1-6 carbon atoms, dihaloalkenyl of 2-7 carbon atoms, —COP, —COCF₃, —CO₂P, —CONR₁R₂, -alkyl-CONR, R₂, -alkenyl-CONR, R₂, -alkyl-COP, -alkenyl-COP, -alkenyl-CO₂P, -alkenyl-CO₂P, oxadiazolyl, furyl, thienyl, pyrrolyl, imidazolyl, triazolyl, or tetrazolyl;
X and Y are each, independently, hydrogen, alkyl of 1-6 carbon atoms, halogen, —NO₂, —CN, trifluoroalkyl of 1-6 carbon atoms, —OP, hydroxyalkyl of 1-6 carbon atoms, —CO₂H, or phenyl which is optionally mono- or di-substituted with hydroxyl, benzyloxy, alkoxy of 1-6 carbon atoms, or —OCH₂CH₂NR₁R₂;
R₁ and R₂ are each, independently, hydrogen, alkyl of 1-6 carbon atoms, or alkoxy of 1-6 carbon atoms; or R₁ and R₂ are concatenated together as —(CH₂)ₚ—;
p=2-6;
or a pharmaceutically acceptable salt or prodrug thereof.

46. The method of claim 32 wherein the ERβ selective ligand has the Formula VII: 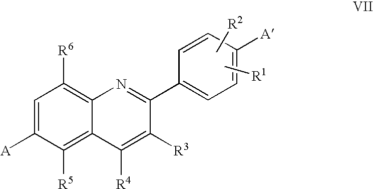
wherein:
A and A′ are each, independently, OH or OP;
P is alkyl, alkenyl, benzyl, acyl, aroyl, alkoxycarbonyl, sulfonyl or phosphoryl;
R¹ and R² are each, independently, H, halogen, C₁-C₆ alkyl, C₂-C₇ alkenyl, or C₁-C₆ alkoxy;
R³ is H, halogen, or C₁-C₆ alkyl;
R⁴ is H, halogen, C₁-C₆ alkyl, C₂-C₇ alkenyl, C₂-C₇ alkynyl, C₃-C₇ cycloalkyl, C₁-C₆ alkoxy, —CN, —CHO, acyl, or heteroaryl;
R⁵ and R⁶ are each, independently, H, halogen, C₁-C₆ alkyl, C₂-C₇ alkenyl, C₂-C₇ alkynyl, C₃-C₇ cycloalkyl; C₁-C₆ alkoxy, —CN, —CHO, acyl, phenyl, aryl or heteroaryl, provided that at least one of R⁴, R⁵ and R⁶ is halogen, C₁-C₆ alkyl, C₂-C₇ alkenyl, C₂-C₇ alkynyl, C₃-C₇ cycloalkyl, C₁-C₆ alkoxy, —CN, —CHO, acyl, phenyl, aryl or heteroaryl;
wherein the alkyl or alkenyl moieties of R⁴, R⁵ or R⁶ may be optionally substituted with halogen, OH, —CN, trifluoroalkyl, trifluoroalkoxy, —NO₂, or phenyl;
wherein the alkynyl moiety of R⁴, R⁵ or R⁶ may be optionally substituted with halogen, —CN, —CHO, acyl, trifluoroalkyl, trialkylsilyl, or optionally substituted phenyl;
wherein the phenyl moiety of R⁵ or R⁶ may be optionally mono-, di-, or tri-substituted with halogen, C₁-C₆ alkyl, C₂-C₇ alkenyl, OH, C₁-C₆ alkoxy, —CN, —CHO, —NO₂, amino, C₁-C₆ alkylamino, di-(C₁-C₆)alkylamino, thiol, or C₁-C₆ alkylthio;
provided that when each of R⁴, R⁵ and R⁶ are H, C₁-C₆ alkyl, C₂-C₇ alkenyl, or C₁-C₆ alkoxy, then at least one of R¹ and R² is halogen, C₁-C₆ alkyl, C₂-C₇ alkenyl, or C₁-C₆ alkoxy;
provided that at least one of R⁴ and R⁶ is other than H;
or a N-oxide thereof;
or Formula VIII: 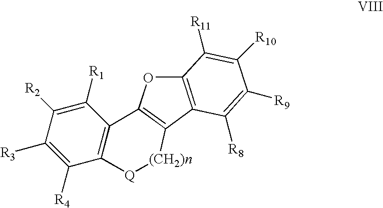
wherein:
Q has the structure i, ii or iii: 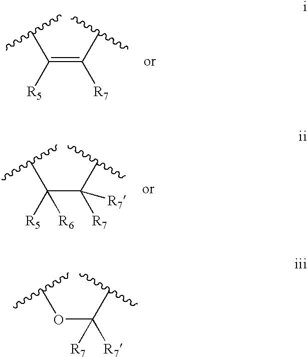
R₁, R₄, R₅, R₆ R₇, R_{7′}, R₈ and R₁₁ are each independently selected from the group consisting of hydrogen, C₁-C₆ alkyl, —OR₂₀, halogen, —CF₃, —CF₂CF₃, —CH₂CF₃, —SR₂₀, NR₂₀R₂₁, —CN, —CH₂CN, —CH₂CH₂CN, —CH═CHCN, —NO₂, —CH₂NO₂, —CH₂CH₂NO₂, —CH═CHNO₂ and —COR₂₀;
n=0 or 1;
each R₂₀ and R₂₁ is independently selected from the group consisting of hydrogen, C₁-C₆ alkyl, —CF₃, benzyl, —CO₂(C₁-C₆ alkyl) and —CO(C₁-C₆ alkyl); provided that:
a) one of R₂ or R₃ must be —OR₂₀;
b) one of R₉ or R₁₀ must be —OR₂₀;
c) when R₂ is —OR₂₀, then R₁ and R₃ are selected independently from the group consisting of hydrogen, halogen, C₁-C₆ alkyl, —CF₃, —CF₂CF₃, —CH₂CF₃, —SR₂₀, —CN, —CH₂CN, —CH₂CH₂CN, —CH═CHCN, —NO₂, —CH₂NO₂, —CH₂CH₂NO₂, —CH═CHNO₂ and —COR₂₀;
d) when R₃ is —OR₂₀, then R₂ and R₄ are selected independently from the group consisting of hydrogen, C₁-C₆ alkyl, halogen, —CF₃, —CF₂CF₃, —CH₂CF₃, —SR₂₀, —CN, —CH₂CN, —CH₂CH₂CN, —CH═CHCN, —NO₂, —CH₂NO₂, —CH₂CH₂NO₂, —CH═CHNO₂ and —COR₂₀;
e) when R₉ is —OR₂₀, then R₈ and R₁₀ are selected independently from the group consisting of hydrogen, C₁-C₆ alkyl, halogen, —CF₃, —CF₂CF₃, —CH₂CF₃, —SR₂₀, —CN, —CH₂CN, —CH₂CH₂CN, —CH═CHCN, —NO₂, —CH₂NO₂, —CH₂CH₂NO₂, —CH═CHNO₂ and —COR₂₀;
f) when R₁₀ is —OR₂₀, then R₉ and R₁₁ are selected independently from the group consisting of hydrogen, C₁-C₆ alkyl, halogen, —CF₃, —CF₂CF₃, —CH₂CF₃, —SR₂₀, —CN, —CH₂CN, —CH₂CH₂CN, —CH═CHCN, —NO₂, —CH₂NO₂, —CH₂CH₂NO₂, —CH═CHNO₂ and —COR₂₀; and
g) when Q has the structure iii, and R₇, R₇, R₈, R₉, R₁₁ are each H, and n=0, then R₁₀ is not OR₂₀,
or a pharmaceutically acceptable salt or prodrug thereof;
or Formula IX: 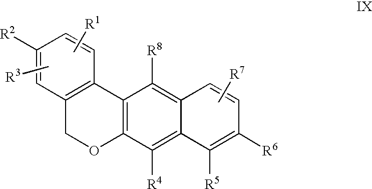
wherein:
R₁, R₂, R₃, R₅, R₆, R₇, and R₈ are each, independently, selected from hydrogen, hydroxyl, C₁-C₆ alkyl, C₁-C₆ alkoxy, or halogen;
R₄ is hydrogen, C₁-C₆ alkyl, halogen, C₁-C₆ alkoxy, —CN, C₂-C₈ alkenyl, —CHO, aryl, furyl, thienyl, pyrimidinyl, or pyridinyl;
provided that at least one of R₁- R₈ is other than H;
or a pharmaceutically acceptable salt or prodrug thereof;
or of Formula X: 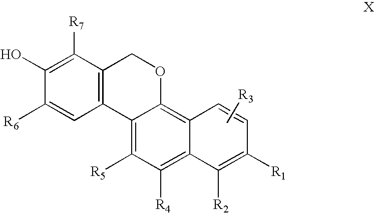
wherein:
R₁ and R₂ are each, independently, selected from hydrogen, hydroxyl, alkyl of 1-6 carbon atoms, alkenyl of 2-6 carbon atoms, alkynyl of 2-7 carbon atoms, alkoxy of 1-6 carbon atoms, or halogen; wherein the alkyl or alkenyl moieties of R₁ or R₂ may be optionally substituted with hydroxyl, —CN, halogen, trifluoroalkyl, trifluoroalkoxy, —NO₂, or phenyl; and provided that at least one of R₁ or R₂ is hydroxyl;
R₃, R₄, R₅, R₆, and R₇ are each, independently, hydrogen, alkyl of 1-6 carbon atoms, halogen, alkoxy of 1-6 carbon atoms, —CN, alkenyl of 2-7 carbon atoms, alkynyl of 2-7 carbon atoms, —CHO, phenyl, or a 5 or 6-membered heterocyclic ring having 1 to 4 heteroatoms selected from O, N or S; wherein the alkyl or alkenyl moieties of R₄, R₅, R₆, or R₇ may be optionally substituted with hydroxyl, —CN, halogen, trifluoroalkyl, trifluoroalkoxy, —NO₂, or phenyl;
wherein the phenyl moiety of R₄ or R₅ may be optionally mono-, di-, or tri-substituted with alkyl of 1-6 carbon atoms, alkenyl of 2-7 carbon atoms, halogen, hydroxyl, alkoxy of 1-6 carbon atoms, —CN, —NO₂, amino, alkylamino of 1-6 carbon atoms, dialkylamino of 1-6 carbon atoms per alkyl group, thio, alkylthio of 1-6 carbon atoms, alkylsulfinyl of 1-6 carbon atoms, alkylsulfonyl of 1-6 carbon atoms, alkoxycarbonyl of 2-7 carbon atoms, alkylcarbonyl of 2-7 carbon atoms, or benzoyl;
or a pharmaceutically acceptable salt or prodrug thereof.

47. The method of claim 32 wherein said disease or disorder is schizophrenia.

48. The method of claim 47 wherein said symptom of schizophrenia is selected from the group consisting of positive, negative, cognitive symptoms.

49. The method of claim 48 wherein said symptom of schizophrenia is a positive symptom.

50. The method of claim 49 wherein said positive symptom is hallucinations, delusions or paranoia.

51. The method of claim 48 wherein said symptom of schizophrenia is a negative symptom.

52. The method of claim 51 wherein said negative symptom is social withdrawal, flat affect, anhedonia or decreased motivation.

53. The method of claim 48 wherein said symptom of schizophrenia is a cognitive symptom.

54. The method of claim 53 wherein said cognitive symptom is a severe deficit in attention, object naming, working memory, long-term memory storage or executive functioning.

55. The method of claim 53 wherein said cognitive symptom comprises long-term memory storage or executive functioning.

56. The method of claim 53 wherein said cognitive symptom is a slowing of information processing, neural activity or long term depression.

57. The method of claim 32 wherein said disease or disorder is multiple sclerosis.

58. The method of claim 57 wherein said symptom of multiple sclerosis is selected from the group consisting of optic neuritis blurred vision, eye pain, loss of color vision, blindness, diplopia double vision, nystagmus jerky eye movements, ocular dysmetria constant under- or overshooting eye movements, internuclear ophthalmoplegia, nystagmus, diplopia, movement and sound phosphenes, nystagmus, diplopia, afferent pupillary defect, motor paresis, monoparesis, paraparesis, hemiparesis, quadraparesis plegia, paraplegia, hemiplegia, tetraplegia, quadraplegia, spasticity, dysarthria, muscle atrophy, spasms, cramps, hypotonia, clonus, myoclonus, myokymia, restless leg syndrome, footdrop dysfunctional reflexes (msrs, babinski's, hoffman's, chaddock's), paraesthesia, anaesthesia, neuralgia, neuropathic and neurogenic pain, I'hermilte's, proprioceptive dysfunction, trigeminal neuralgia, ataxia, intention tremor, dysmetria, vestibular ataxia, vertigo, speech ataxia, dystonia, dysdiadochokinesia, frequent micturation, bladder spasticity, flaccid bladder, detrusor-sphincter dyssynergia, erectile dysfunction, anorgasmy, retrograde ejaculation, frigidity, constipation, fecal urgency, depression, cognitive dysfunction, dementia, mood swings, emotional lability, euphoria, bipolar syndrome, anxiety, aphasia, dysphasia, fatigue, uhthoffs symptom, gastroesophageal reflux and sleeping disorders.

59. The method of claim 32 wherein said disease or disorder is depression.

60. The method of claim 59 wherein said symptom of depression is selected from depressed feeling or mood, loss of interest or pleasure in some or all activities, changes in appetite, weight or sleep patterns, lack of energy, fatigue, low self esteem, diminished capacity for thinking, concentration, or decisiveness, feelings of hopelessness or worthlessness, psychomotor. agitation or retardation, self-reproach, inappropriate guilt, frequent thoughts of death or suicide, plans or attempts to commit suicide.

61. The method of claim 32 wherein the disease or disorder is Alzheimer's disease.

62. The method of claim 61 wherein the symptom of Alzheimer's disease is selected from the group consisting of impairment in memory, attention, judgment, decision-making, orientation to physical surroundings, language, speed-dependent activities, abstract reasoning, visuospatial abilities, executive functioning, and behavioral disturbances, disinterest and passivity, apathy, inappropriate dressing, poor self care, agitation, violent outbursts, aggression, depression, anxiety, hallucinations, delusions, changes in personality and mood changes and dementia.

63. The method of claim 32 wherein the disease or disorder is anxiety.

64. The method of claim 63 wherein the symptom of anxiety is selected from the group consisting of apprehension, fear, trembling, muscle aches, insomnia, abdominal upsets, dizziness, irritability, persistent, recurring thoughts, compulsions, heart palpitations, chest pain, chest discomfort, sweating, tingling sensations, feeling of choking, fear of losing control, flashbacks, nightmares, intrusive thoughts, intrusive recollections, avoidance behaviors, emotional numbing, an inability to sleep, anxious feelings, overactive startle response, hypervigilance, outbursts of anger, faintness, blushing and profuse sweating.

65. The method of claim 32 wherein the disease or disorder is stroke.

66. The method of claim 65 wherein the symptom of stroke is selected from the group consisting of hemiparesis, vertigo, numbness, aphasia, dysarthria, dysphasia, facial drooping, loss of balance or coordination, inability to walk, changes in sensation, changes in vision, headache, facial pain, limb pain, disorientation, change in consciousness, chest pain, shortness of breath, palpitations, hiccups, nausea and general weakness.
